SEQUENCE LISTING (42 sequences)

SEQ ID NO: 1 - protein, 11 aa, synthetic construct
  REGION 1..11: note = L-CDR
KASQDINSYLT

SEQ ID NO: 2 - protein, 7 aa, synthetic construct
  REGION 1..7: note = L-CDR
LVNRLVD

SEQ ID NO: 3 - protein, 9 aa, synthetic construct
  REGION 1..9: note = L-CDR
LQYDDFPYT

SEQ ID NO: 4 - protein, 5 aa, synthetic construct
  REGION 1..5: note = H-CDR
THAMS

SEQ ID NO: 5 - protein, 17 aa, synthetic construct
  REGION 1..17: note = H-CDR
TISSGGRSTYYPDSVKG

SEQ ID NO: 6 - protein, 5 aa, synthetic construct
  REGION 1..5: note = H-CDR
LISTY

SEQ ID NO: 7 - protein, 7 aa, synthetic construct
  REGION 1..7: note = L-CDR
LTNRLMD

SEQ ID NO: 8 - protein, 9 aa, synthetic construct
  REGION 1..9: note = L-CDR
LQYVDFPYT

SEQ ID NO: 9 - protein, 5 aa, synthetic construct
  REGION 1..5: note = H-CDR
SSAMS

SEQ ID NO: 10 - protein, 5 aa, synthetic construct
  REGION 1..5: note = H-CDR
LISPY

SEQ ID NO: 11 - protein, 7 aa, synthetic construct
  REGION 1..7: note = L-CDR
RTNRLFD

SEQ ID NO: 12 - protein, 5 aa, synthetic construct
  REGION 1..5: note = H-CDR
LISLY

SEQ ID NO: 13 - protein, 107 aa, synthetic construct
  REGION 1..107: note = Light Chain
DIQMTQTPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 14 - protein, 107 aa, synthetic construct
  REGION 1..107: note = Light Chain of AB1 antibody
DIKMTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 15 - protein, 107 aa, synthetic construct
  REGION 1..107: note = Light Chain
EIVLTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 16 - protein, 107 aa, synthetic construct
  REGION 1..107: note = Light Chain
DIQMTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 17 - protein, 107 aa, synthetic construct
  REGION 1..107: note = Light Chain
EIVLTQSPSSLSASVGDRVTITCKASQDINSYLTWYQQKPGKAPKLLIYRTNRLFDGVPSRFSGSGSGTDFFFTISSLQPEDFGTYYCLQYDDFPYTFGGGTKLEIK

SEQ ID NO: 18 - protein, 107 aa, synthetic construct
  REGION 1..107: note = Light Chain
DIQMTQSPSSLSASVGDRVTITCKASQDINSYLTWFQQKPGKAPKSLIYRTNRLFDGVPSRFSGSGSGTDFFLTISSLQPEDFATYYCLQYDDFPYTFGQGTKVEIK

SEQ ID NO: 19 - protein, 107 aa, synthetic construct
  REGION 1..107: note = Light Chain of BB7 antibody
AIKMTQSPSSMYASLGERVIITCKASQDINSYLTWFQQKPGKSPKTLIYLTNRLMDGVPSRFSGSGSGQEFLLTISGLEHEDMGIYYCLQYVDFPYTFGGGTKLEIK

SEQ ID NO: 20 - protein, 107 aa, synthetic construct
  REGION 1..107: note = Light Chain
DIQMTQSPSSLSASVGDRVTITCKASQDINSYLTWFQQKPGKAPKSLIYLTNRLMDGVPSRFSGSGSGTDFFLTISSLQPEDFATYYCLQYVDFPYTFGQGTKVEIK

SEQ ID NO: 21 - protein, 107 aa, synthetic construct
  REGION 1..107: note = Light Chain
DIKMTQSPSSLSASVGDRVTITCKASQDINSYLTWFQQKPGKAPKTLIYLTNRLMDGVPSRFSGSGSGQEFLLTISSLQPEDFATYYCLQYVDFPYTFGQGTKVEIK

SEQ ID NO: 22 - protein, 107 aa, synthetic construct
  REGION 1..107: note = Light Chain of DC1 antibody
DITMTQSPSSIYASLGERVTITCKASQDINSYLTWFQQKPGKSPKILIYLVNRLVDGVPSRFSGSGSGQDYALTISSLEYEDMGIYYCLQYDDFPYTFGGGTKLEIK

SEQ ID NO: 23 - protein, 107 aa, synthetic construct
  REGION 1..107: note = Light Chain
DIQMTQSPSSLSASVGDRVTITCKASQDINSYLTWFQQKPGKAPKSLIYLVNRLVDGVPSRFSGSGSGTDFFLTISSLQPEDFATYYCLQYDDFPYTFGQGTKVEIK

SEQ ID NO: 24 - protein, 107 aa, synthetic construct
  REGION 1..107: note = Light Chain of Zampilimab
DITMTQSPSSLSASVGDRVTITCKASQDINSYLTWFQQKPGKAPKILIYLVNRLVDGVPSRFSGSGSGQDYALTISSLQPEDFATYYCLQYDDFPYTFGQGTKVEIK

SEQ ID NO: 25 - protein, 107 aa, synthetic construct
  REGION 1..107: note = Light Chain of rbBB7 antibody
AIKMTQTPSSVSAAVGGTVTISCKASQDINSYLTWFQQKPGQPPKTLIYLTNRLMDGVPSRFSGSGSGQEFLLTISGLECDDAATYYCLQYVDFPYTFGGGTKVVVK

SEQ ID NO: 26 - protein, 112 aa, synthetic construct
  REGION 1..112: note = Light Chain of IA12 antibody
DVVNTQTPLTLSVTFGQPASISCKSSQSLLYDNGKTYLHWLFQRPGQSPRRLIYLVSKLDSGVPDRFTGSGSGTDFTLKISRVEAEDLGVYYCVQGTHFPYTFGGGTKLEIK

SEQ ID NO: 27 - protein, 106 aa, synthetic construct
  REGION 1..106: note = Light Chain
QIVLTQSPAIMSASPGEKVTMTCSASSSVSYMYWYQQKPGSSPRLLIYDTSNLASGVPVRFSGSGSGTSYSLTISRMEAEDAATFYCQQWSSSPLTFGAGTKLELK

SEQ ID NO: 28 - protein, 114 aa, synthetic construct
  REGION 1..114: note = Heavy Chain
EVQLEESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 29 - protein, 114 aa, synthetic construct
  REGION 1..114: note = Heavy Chain of AB1 antibody
EVQLVESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 30 - protein, 114 aa, synthetic construct
  REGION 1..114: note = Heavy Chain
EVQLQESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 31 - protein, 114 aa, synthetic construct
  REGION 1..114: note = Heavy Chain
EVQLLESGGGLVQPGGSLRLSCAASGFTFSSSAMSWVRQAPGKGLEWVSTISVGGGKTYYPDSVKGRFTISRDNSKNTLYLQMNSLRAEDTAVYYCAKLISLYWGQGTLVTVSS

SEQ ID NO: 32 - protein, 114 aa, synthetic construct
  REGION 1..114: note = Heavy Chain of BB7 antibody
AVQLVESGGGLVKPGGSLKLSCAASGIIFSSSAMSWVRQTPEKRLEWVATISSGGRSTYYPDSVKGRFTVSRDSAKNTLYLQMDSLRSEDTAIYYCAKLISPYWGQGTTLTVSS

SEQ ID NO: 33 - protein, 114 aa, synthetic construct
  REGION 1..114: note = Heavy Chain
EVQLLESGGGLVQPGGSLRLSCAASGFTFSSSAMSWVRQAPGKGLEWVSTISSGGRSTYYPDSVKGRFTISRDNSKNTLYLQMNSLRAEDTAVYYCAKLISPYWGQGTLVTVSS

SEQ ID NO: 34 - protein, 114 aa, synthetic construct
  REGION 1..114: note = Heavy Chain
EVQLLESGGGLVQPGGSLRLSCAASGIIFSSSAMSWVRQAPGKGLEWVATISSGGRSTYYPDSVKGRFTVSRDSSKNTLYLQMNSLRAEDTAVYYCAKLISPYWGQGTLVTVSS

SEQ ID NO: 35 - protein, 114 aa, synthetic construct
  REGION 1..114: note = Heavy Chain of DC1 antibody
EVQLVESGGGLVKPGGSLKLSCAASGFTLSTHAMSWVRQTPEKRLEWVATISSGGRSTYYPDSVKGRFTISRDNVKNTLYLQLSSLRSEDTAVYFCARLISTYWGQGTTLTVSS

SEQ ID NO: 36 - protein, 114 aa, synthetic construct
  REGION 1..114: note = Heavy Chain
EVQLLESGGGLVQPGGSLRLSCAASGFTFSTHAMSWVRQAPGKGLEWVSTISSGGRSTYYPDSVKGRFTISRDNSKNTLYLQMNSLRAEDTAVYYCAKLISTYWGQGTLVTVSS

SEQ ID NO: 37 - protein, 114 aa, synthetic construct
  REGION 1..114: note = Heavy Chain of Zampilimab
EVQLLESGGGLVQPGGSLRLSCAASGFTLSTHAMSWVRQAPGKGLEWVATISSGGRSTYYPDSVKGRFTISRDNSKNTLYLQMNSLRAEDTAVYFCARLISTYWGQGTLVTVSS

SEQ ID NO: 38 - protein, 114 aa, synthetic construct
  REGION 1..114: note = Heavy Chain of rbBB7 antibody
EVQLLESGGRLVTPGGSLTLTCAASGIIFSSSAMSWVRQAPGKGLEWVATISSGGRSTYYPDSVKGRFTVSRDSSKNTLYLQMNSLRAEDTAVYYCAKLISPYWGQGTLVTVSS

SEQ ID NO: 39 - protein, 114 aa, synthetic construct
  REGION 1..114: note = Heavy Chain of IA12 antibody
QIQLVQSGPELKKPGETVKISCKASGYTFTTYGNTWVKQAPGKGLKWMGWINTSSGVPTYADDFKGRFAFSLETSASTAYLQINNLKSEDTATYFCARPEVAYWGQGTLVTVSA

SEQ ID NO: 40 - protein, 119 aa, synthetic construct
  REGION 1..119: note = Heavy Chain
QVTLKESGPGILQPSQTLSLTCSFSGFSLSTSGMGVSWIRQPSGKGLEWLAHIYWDDDKRYNPSLKSRLTISKDTSSNQVFLKITSVDTADTATYYCARSGTTAPFAYWGQGTLVTVSA

SEQ ID NO: 41 - protein, 686 aa, Homo sapiens
  REGION 1..686: note = Human TG2
AEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 42 - protein, 17 aa, synthetic construct
TISVGGGKTYYPDSVKG

(19) United States
(12) Patent Application Publication
(10) Pub. No.: US 2025/0026856 A1
(43) Pub. Date: Jan. 23, 2025

(54) METHOD FOR THE TREATMENT OF A SCLERODERMA DISEASE

(71) Applicant: UCB BIOPHARMA SRL, Brussels (BE)

(72) Inventors: Timothy Scott JOHNSON, Berkshire (GB); John ATKINSON, Berkshire (GB); Jorg Hans Wilhelm DISTLER, Erlangen (DE); David John ABRAHAM, London (GB)

(21) Appl. No.: 18/711,331

(22) PCT Filed: Nov. 17, 2022
(86) PCT No.: PCT/EP2022/082293

§ 371 Date: May 17, 2024

(30) Foreign Application Priority Data
Nov. 18, 2021 (GB) 2116665.7

(51) Int. Cl. C07K 16/40; A61P 17/00

(52) U.S. Cl. CPC C07K 16/40; A61P 17/00; C07K 2317/55; C07K 2317/56

(57) ABSTRACT

The present invention relates to an anti-transglutaminase type 2 (TG2) antibody for use in the treatment of a scleroderma disease, such as localized or systemic scleroderma.

### FIELD OF INVENTION

The present invention relates to an anti-transglutaminase type 2 (TG2) antibody that blocks transamidase activity of the enzyme for use in the treatment of a scleroderma disease, such as localized or systemic scleroderma.

### BACKGROUND OF THE INVENTION

Tissue transglutaminase or transglutaminase type 2 (TG2) is an enzyme which forms crosslinks between proteins via epsilon(gamma-glutamyl) lysine di-peptide bonds. Elevated expression of TG2 leads to aberrant protein cross-linking which has been associated with several pathologies including various types of tissue scarring, the formation of neurofibrillary tangles in several brain disorders and resistance to chemotherapy in some cancers. Various TG2 inhibitors, such as small molecules, silencing RNA or antibodies (e.g. Siegel 2007, Wang 2020, WO2006100679, WO2012146901 or WO2013175229), have been disclosed for the possible treatment of TG2-mediated disorders.

Scleroderma (also called Systemic sclerosis) is an immune-mediated rheumatic disease, mainly characterised by fibrosis of the skin and internal organs as well as vasculopathy (Careta & Romiti 2015; Denton & Khanna, 2017). Two categories of scleroderma have been identified so far: systemic sclerosis (SSc) and localized scleroderma (LoS) (Careta & Romiti 2015). SSc is characterized by cutaneous sclerosis and visceral involvement typically limited to the skin and/or underlying tissues. LoS is a chronic connective tissue disease having different clinical manifestations depending on the LoS subtypes.

Immunosuppressive treatments have a central role for the treatment of SSc currently, with cyclophosphamide remaining the first choice for treatment of SSc interstitial lung disease (ILD), successful in stabilising respiratory functions. However, there are none negligible side effects and the benefits are not maintained after the cessation of the therapy (Barsotti 2019).

Therefore, there remains a need to identify further effective therapies for use in treatment and prevention of scleroderma diseases, such as localized or systemic scleroderma.

### SUMMARY OF THE INVENTION

It is an object of the present invention to provide an anti-Transglutaminase type 2 (TG2) antibody for use in the treatment of a scleroderma disease or for use in the prevention of the development of a scleroderma disease. Preferably, the scleroderma disease is localized scleroderma, systemic scleroderma or systemic scleroderma with interstitial lung disease.

In a second aspect, the invention provides a method for treating or for preventing the development of a scleroderma disease comprising administering a therapeutically effective amount of an anti-TG2 antibody.

In a third aspect, the invention relates to the use of an anti-Transglutaminase type 2 (TG2) antibody for the manufacturing of a medicament in the treatment of a scleroderma disease or for the prevention of the development of a scleroderma disease.

### Definitions

The entire document is intended to be related as a unified disclosure, and it should be understood that all combinations of features described herein are contemplated, even if the combination of features are not found together in the same sentence, or paragraph, or section of this document. With respect to aspects of the invention described or claimed with “a” or “an”, it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.
The term “Tissue transglutaminase”, “Transglutaminase type 2” or “TG2” refers to an enzyme which forms crosslinks between proteins via epsilon(gamma-glutamyl) lysine di-peptide bonds. TG2 refers to a protein that typically has the amino acid sequence as set out in the UniProt entry P21980 (SEQ ID NO: 41), i.e. human TG2. The term “TG2” may also refer to protein which is (a) a derivative having one or more amino acid substitutions, modifications, deletions or insertions relative to the amino acid sequence of SEQ ID NO: 41 which retains the activity of TG2, or (b) a variant thereof, such variants typically retain at least about 75%, 80%, 85%, 90%, 91%, 92%, 93%, 94% or 95% identity to SEQ ID NO: 41 (or even about 96%, 97%, 98% or 99% identity to SEQ ID NO: 41).
The term “anti-TG2 antibody”, as used herein, is intended to be an antibody molecule which binds TG2 and block its transamidase activity to prevent crosslinking. Examples of such antibodies are described in WO2013175229. Without any limitation, an anti-TG2 antibody that can be used according to the present invention comprises for instance a light chain variable region as defined in SEQ ID NO: 24 and a heavy chain variable region as defined in SEQ ID NO: 37.
The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments, more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv)2 (also referred to as TrYbe®, see WO2015/197772 for instance). Antibody molecules as defined above, including antigen-binding fragments thereof, are known in the art.
The term “epitope” refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.
The term “treating” or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.
The term “preventing” or “prevention” of a disease state includes causing the clinical symptoms of the disease state not to develop in a subject that may be exposed to or predisposed to the disease state, but does not yet experience or display symptoms of the disease state.

### DETAILED DESCRIPTION OF THE INVENTION

The invention is based on the finding from the inventor that total amount of TG2 protein was increased in the skin and the lung fibroblasts of SSc subjects (in particular, expression levels and the activity of TG2 were increased in SSc fibroblasts), with higher levels in SSc subjects with pulmonary involvement. In contrast, the circulating levels of TG2 in serum and plasma are low and detectable only in a subset of SSc subjects and controls. The inventors were then able to surprisingly demonstrate that anti-TG2 antibodies could attenuate extracellular matrix (ECM) deposition in a subset of SSc fibroblasts not only under standard 2D culture conditions, but also under 3D culture conditions in full thickness skin.

The main object of the present invention is an anti-Transglutaminase type 2 (TG2) antibody for use in the treatment of a scleroderma disease or for use in the prevention of the development of a scleroderma disease. Preferably, the scleroderma disease is localized scleroderma or systemic scleroderma. The scleroderma disease can also be systemic scleroderma with interstitial lung disease. For instance, the anti-TG2 antibody for use according to the invention can comprises the following sequences:

```
(i)
(LCDR1; SEQ ID NO: 1)
KASQDINSYLT;
(LCDR2; SEQ ID NO: 2)
LVNRLVD;
(LCDR3; SEQ ID NO: 3)
LQYDDFPYT;
  (HCDR1; SEQ ID NO: 4)
THAMS;
(HCDR2; SEQ ID NO: 5)
TISSGGRSTYYPDSVKG;
and
(HCDR3; SEQ ID NO: 6)
LISTY;
or
(ii)
(LCDR1; SEQ ID NO: 1)
KASQDINSYLT;
(LCDR2; SEQ ID NO: 7)
LTNRLMD;
(LCDR3; SEQ ID NO: 8)
LQYVDFPYT;
    (HCDR1; SEQ ID NO: 9)
SSAMS;
(HCDR2; SEQ ID NO: 5)
TISSGGRSTYYPDSVKG;
and
(HCDR3; SEQ ID NO: 10)
LISPY;
(iii)
(LCDR1; SEQ ID NO: 1)
KASQDINSYLT;
(LCDR2; SEQ ID NO: 11)
RTNRLFD;
  (LCDR3; SEQ ID NO: 3)
LQYDDFPYT;
SSAMS (HCDR1);
(HCDR2; SEQ ID NO: 9)
TISVGGGKTYYPDSVKG;
and
(HCDR3; SEQ ID NO: 12)
LISLY,
```

(iv) compete with an antibody according to any one of (i) to (iii).

The invention also provides a method for the treatment or for the prevention of the development of a scleroderma disease comprising administering a therapeutically effective amount of an anti-TG2 antibody. Preferably, the scleroderma disease is localized scleroderma or systemic scleroderma. The scleroderma disease can also be systemic scleroderma with interstitial lung disease. For instance, the anti-TG2 for use according to the invention can comprises the following sequences:

```
(i)
(LCDR1; SEQ ID NO: 1)
KASQDINSYLT;
(LCDR2; SEQ ID NO: 2)
LVNRLVD;
(LCDR3; SEQ ID NO: 3)
LQYDDFPYT;
    (HCDR1; SEQ ID NO: 4)
THAMS;
(HCDR2; SEQ ID NO: 5)
TISSGGRSTYYPDSVKG;
and
(HCDR3; SEQ ID NO: 6)
LISTY;
or
(ii)
(LCDR1; SEQ ID NO: 1)
KASQDINSYLT;
(LCDR2; SEQ ID NO: 7)
LTNRLMD;
(LCDR3; SEQ ID NO: 8)
LQYVDFPYT;
    (HCDR1; SEQ ID NO: 9)
SSAMS;
(HCDR2; SEQ ID NO: 5)
TISSGGRSTYYPDSVKG;
and
(HCDR3; SEQ ID NO: 10)
LISPY;
(iii)
(LCDR1; SEQ ID NO: 1)
KASQDINSYLT;
(LCDR2; SEQ ID NO: 11)
RTNRLFD;
  (LCDR3; SEQ ID NO: 3)
LQYDDFPYT;
(HCDR2; SEQ ID NO: 9)
SSAMS (HCDR1);
TISVGGGKTYYPDSVKG;
and
(HCDR3; SEQ ID NO: 12)
LISLY,
```

(iv) compete with an antibody according to any one of (i) to (iii).

Also described is the use of an anti-Transglutaminase type 2 (TG2) antibody for the manufacturing of a medicament in the treatment of a scleroderma disease or for the preventing the development of a scleroderma disease. Preferably, the scleroderma disease is localized scleroderma or systemic scleroderma. The scleroderma disease can also be systemic scleroderma with interstitial lung disease. For instance, the anti-TG2 for use according to the invention can comprises the following sequences:

(i)
(LCDR1; SEQ ID NO: 1)
KASQDINSYLT;
(LCDR2; SEQ ID NO: 2)
LVNRLVD;
(LCDR3; SEQ ID NO: 3)
LQYDDFPYT;
(HCDR1; SEQ ID NO: 4)
THAMS;
(HCDR2; SEQ ID NO: 5)
TISSGGRSTYYPDSVKG;
and
(HCDR3; SEQ ID NO: 6)
LISTY;
or
(ii)
(LCDR1; SEQ ID NO: 1)
KASQDINSYLT;
(LCDR2; SEQ ID NO: 7)
LTNRLMD;
(LCDR3; SEQ ID NO: 8)
LQYVDFPYT;
(HCDR1; SEQ ID NO: 9)
SSAMS;
(HCDR2; SEQ ID NO: 5)
TISSGGRSTYYPDSVKG;
and
(HCDR3; SEQ ID NO: 10)
LISPY;
(iii)
(LCDR1; SEQ ID NO: 1)
KASQDINSYLT;
(LCDR2; SEQ ID NO: 11)
RTNRLFD;
(LCDR3; SEQ ID NO: 3)
LQYDDFPYT;
(HCDR2; SEQ ID NO: 9)
SSAMS (HCDR1);
TISVGGGKTYYPDSVKG;
and
(HCDR3; SEQ ID NO: 12)
LISLY,

(iv) compete with an antibody according to any one of (i) to (iii).

In the context of the present invention as a whole, the scleroderma disease is characterised by an increase of a marker in a subject's sample, wherein the marker is for instance any one of TG2 expression or TG2 activity, and wherein the subject's sample is a cell or a tissue associated with said disease (e.g. fibroblasts or keratinocytes). The increase of TG2 expression (alternatively called overexpression of TG2) may be determined by any means in fibroblasts or keratinocytes for instance. An increase of a marker in one subject's sample is typically determined by comparison of the level of said marker in the subject's sample to the level of the same marker in normal cells of the same tissue type (i.e. basal level; e.g. basal TG2 activity, basal expression level (mRNA level and/or protein level). A subject's sample having a level of at least one marker equal or higher than 10%, equal or higher than 15%, equal or higher than 20%, equal or higher than 25% or even equal or higher than 30% compared to the basal level for said marker will be consider as presenting an increase of said marker. For example, an increase of TG2 expression (alternatively called TG2 overexpression) can be determined via determination of the amount of TG2 mRNA in dermal fibroblasts or keratinocytes of a subject. Scleroderma disease cells/tissues may thus be characterised for instance by an increased amount (representing overexpression) of TG2 mRNA in dermal fibroblasts or keratinocytes of a subject, compared with normal dermal fibroblasts or keratinocytes. The expression of TG2 mRNA may be increased by any amount, such as equal or higher than 10%, equal or higher than 15%, equal or higher than 20%, equal or higher than 25% or even equal or higher than 30%% compared to the basal level. The amount of mRNA can be measured using any known methods such as quantitative reverse transcription polymerase chain reaction (qRT-PCR), real time qRT-PCR, quantigene assay (Affymetrix/Thermo Fisher), by northern blotting or using microarrays, RNA sequencing and various types of in situ hybridisation (e.g. RNAscope). Alternatively, overexpression can be determined via determination of the amount of TG2 antigen in some cells of a subject, for instance in dermal fibroblasts, keratinocytes or lung cells. The sclerodermic cells may thus be characterised for instance by an increased amount (representing overexpression) of TG2 protein (or TG2 antigen) in dermal fibroblasts or keratinocytes of a subject, such as compared with normal dermal fibroblasts or keratinocytes. The expression of TG2 protein may be increased by any amount, such as equal or higher than 10%, equal or higher than 15%, equal or higher than 20%, equal or higher than 25% or even equal or higher than 30% compared to the basal level. The amount of protein can be measured using any known methods such as immunohistochemistry, western blotting, mass spectrometry or fluorescence-activated cell sorting (FACS), including by use of an anti-TG2 antibody of the invention. The thresholds for determining expression may vary depending on the techniques that are used and may be validated against immunohistochemistry scores. Alternatively, the sclerodermic cells may be characterised by an increase of the TG2 activity in dermal fibroblasts or keratinocytes of a subject, compared with normal cells of the same tissue type. TG2 activity may be increased by any amount, such as equal or higher than 10%, equal or higher than 15%, equal or higher than 20%, equal or higher than 25% or even equal or higher than 30% compared to the basal level. TG2 activity can be measured using any known methods such as via biopsy.

The anti-TG2 antibodies for use, method for treating or use of anti-TG2 according to the invention, i.e. for treating or preventing scleroderma disease in a subject, may thus comprise the steps of (a) measuring TG2 expression or TG2 activity in a sample (e.g. fibroblasts (such as dermal fibroblasts) or keratinocytes) from the subject, (b) comparing the result of the measure obtained from a) to the corresponding measure in a normal cell/tissue (e.g. fibroblasts (such as dermal fibroblasts) or keratinocytes), and c) if an increase of expression (i.e. overexpression of TG2) or an increase of activity is observed, administering to the subject an anti-TG2 antibody, thereby treating or preventing the scleroderma disease. TG2 expression that is measured in step a) can be the mRNA or protein amount, and the increase can be any increase of expression as discussed above. The corresponding measure in a normal cell/tissue (e.g. fibroblasts (such as dermal fibroblasts) or keratinocytes) does not need to be obtained each time a comparison is to be made. Said corresponding measure can be obtained any time before the comparison is to be made and can be the average TG2 expression or TG2 activity in said normal cell/tissue (e.g. fibroblasts (such as dermal fibroblasts) or keratinocytes).

In the context of the invention as a whole, the anti-TG2 antibody binds to an epitope within the core region of human transglutaminase type 2 (TG2) and inhibits human TG2 activity, wherein said core region consists of amino acids 143 to 473 of human TG2, and wherein the human TG2 activity that is inhibited is the TG2 cross-linking of lysine and glutamine with Nε(γ-glutamyl)lysine isopeptide bonds. Even preferably, the antibody binds to region comprising or consisting of amino acids 304 to 326 of human TG2 or part of this region. Said antibody can comprise or consist of an intact antibody. Alternatively it can comprise or consist of an antigen-binding fragment such as (but not limited to) an Fv fragment (for example a single chain Fv fragment or a disulphide-bonded Fv fragment); a Fab fragment; and a Fab-like fragment (for example an Fab′ fragment or an F(ab)2 fragment). Preferably, the anti-TG2 antibody to be used according to the invention as a whole (see also Table A):
a) comprises 6 CDRs selected from the group consisting of:

(i)
(LCDR1; SEQ ID NO: 1)
KASQDINSYLT;
(LCDR2; SEQ ID NO: 2)
LVNRLVD;
(LCDR3; SEQ ID NO: 3)
LQYDDFPYT;
(HCDR1; SEQ ID NO: 4)
THAMS;
(HCDR2; SEQ ID NO: 5)
TISSGGRSTYYPDSVKG;
and
(HCDR3; SEQ ID NO: 6)
LISTY;
(ii)
(LCDR1; SEQ ID NO: 1)
KASQDINSYLT;
(LCDR2; SEQ ID NO: 7)
LTNRLMD;
(LCDR3; SEQ ID NO: 8)
LQYVDFPYT;
(HCDR1; SEQ ID NO: 9)
SSAMS;
(HCDR2; SEQ ID NO: 5)
TISSGGRSTYYPDSVKG;
and
(HCDR3; SEQ ID NO: 10)
LISPY;
or
(iii)
(LCDR1; SEQ ID NO: 1)
KASQDINSYLT;
(LCDR2; SEQ ID NO: 11)
RTNRLFD;
(LCDR3; SEQ ID NO: 3)
LQYDDFPYT;
(HCDR2; SEQ ID NO: 9)
SSAMS (HCDR1);
TISVGGGKTYYPDSVKG;
and
(HCDR3; SEQ ID NO: 12)
LISLY.

b) comprises a light chain variable domain having the sequence as defined in any one of SEQ ID NO: 13 to SEQ ID NO: 27 and a heavy chain variable domain having the sequence as defined in any one of SEQ ID NO: 28 to SEQ ID NO: 40,
c) comprises a light chain variable domain having at least 80% identity or similarity, preferably at least 90% identity or similarity, preferably at least 95% identity or similarity to the sequence as defined in any one of SEQ ID NO: 13 to SEQ ID NO: 27 and a heavy chain variable domain having at least 80% identity or similarity, preferably at least 90% identity or similarity, or preferably at least 95% identity or similarity to the sequence as defined in any one of SEQ ID NO: 28 to SEQ ID NO: 40,
d) compete for binding to an epitope comprising or consisting of amino acids 304 to 326 of human TG2 (SEQ ID NO:41) or part of this region with an antibody as defined in a), b) or c) above.

TABLE A
TG2 and Anti-TG2 amino acid sequences

| SEQ | |
|---|---|
| ID | Amino acid sequence |
| 1 | KASQDINSYLT |
| 2 | LVNRLVD |
| 3 | LQYDDFPYT |
| 4 | THAMS |
| 5 | TISSGGRSTYYPDSVKG |
| 6 | LISTY |
| 7 | LTNRLMD |
| 8 | LQYVDFPYT |
| 9 | SSAMS |
| 10 | LISPY |
| 11 | RTNRLFD |
| 12 | LISLY |
| 13 | DIQMTQTPSSMYASLGERVTITCKASQDINSYLTWFQQKPGKSPKTLIYRTNRLFD |
| | GVPSRFSGSGSGQDFFLTISSLEYEDMGIYYCLQYDDFPYTFGGGTKLEIK |
| 14 | DIKMTQSPSSMYASLGERVTITCKASQDINSYLTWFQQKPGKSPKTLIYRTNRLFD |
| | GVPSRFSGSGSGQDFFLTISSLEYEDMGIYYCLQYDDFPYTFGGGTKLEIK |
| 15 | EIVLTQSPSSMYASLGERVTITCKASQDINSYLTWFQQKPGKSPKTLIYRTNRLFDG |
| | VPSRFSGSGSGQDFFLTISSLEYEDMGIYYCLQYDDFPYTFGGGTKLEIK |
| 16 | DIQMTQSPSSMYASLGERVTITCKASQDINSYLTWFQQKPGKSPKTLIYRTNRLFD |
| | GVPSRFSGSGSGQDFFLTISSLEYEDMGIYYCLQYDDFPYTFGGGTKLEIK |
| 17 | EIVLTQSPSSLSASVGDRVTITCKASQDINSYLTWYQQKPGKAPKLLIYRTNRLFDG |
| | VPSRFSGSGSGTDFFFTISSLQPEDFGTYYCLQYDDFPYTFGGGTKLEIK |
| 18 | DIQMTQSPSSLSASVGDRVTITCKASQDINSYLTWFQQKPGKAPKSLIYRTNRLFD |
| | GVPSRFSGSGSGTDFFLTISSLQPEDFATYYCLQYDDFPYTFGQGTKVEIK |
| 19 | AIKMTQSPSSMYASLGERVIITCKASQDINSYLTWFQQKPGKSPKTLIYLTNRLMDG |
| | VPSRFSGSGSGQEFLLTISGLEHEDMGIYYCLQYVDFPYTFGGGTKLEIK |
| 20 | DIQMTQSPSSLSASVGDRVTITCKASQDINSYLTWFQQKPGKAPKSLIYLTNRLMD |
| | GVPSRFSGSGSGTDFFLTISSLQPEDFATYYCLQYVDFPYTFGQGTKVEIK |
| 21 | DIKMTQSPSSLSASVGDRVTITCKASQDINSYLTWFQQKPGKAPKTLIYLTNRLMD |
| | GVPSRFSGSGSGQEFLLTISSLQPEDFATYYCLQYVDFPYTFGQGTKVEIK |
| 22 | DITMTQSPSSIYASLGERVTITCKASQDINSYLTWFQQKPGKSPKILIYLVNRLVDGV |
| | PSRFSGSGSGQDYALTISSLEYEDMGIYYCLQYDDFPYTFGGGTKLEIK |
| 23 | DIQMTQSPSSLSASVGDRVTITCKASQDINSYLTWFQQKPGKAPKSLIYLVNRLVD |
| | GVPSRFSGSGSGTDFFLTISSLQPEDFATYYCLQYDDFPYTFGQGTKVEIK |
| 24 | DITMTQSPSSLSASVGDRVTITCKASQDINSYLTWFQQKPGKAPKILIYLVNRLVDGVPS |
| | RFSGSGSGQDYALTISSLQPEDFATYYCLQYDDFPYTFGQGTKVEIK |
| 25 | AIKMTQTPSSVSAAVGGTVTISCKASQDINSYLTWFQQKPGQPPKTLIYLTNRLMDGVP |
| | SRFSGSGSGQEFLLTISGLECDDAATYYCLQYVDFPYTFGGGTKVVVK |
| 26 | DVVNTQTPLTLSVTFGQPASISCKSSQSLLYDNGKTYLHWLFQRPGQSPRRLIYLVSKL |
| | DSGVPDRFTGSGSGTDFTLKISRVEAEDLGVYYCVQGTHFPYTFGGGTKLEIK |
| 27 | QIVLTQSPAIMSASPGEKVTMTCSASSSVSYMYWYQQKPGSSPRLLIYDTSNLASGVP |
| | VRFSGSGSGTSYSLTISRMEAEDAATFYCQQWSSSPLTFGAGTKLELK |
| 28 | EVQLEESGGGLVKPGGSLKLSCAASGFTLSSSAMSWVRQTPDRRLEWVATISVG |
| | GGKTYYPDSVKGRFTISRDNAKNTLYLQMNSLRSEDTAMYYCAKLISLYWGQGTT |
| | LTVSS |
| 29 | EVQLVESGGGLVKPGGSLKLSCAASGFTLSSSAMSWVRQTPDRRLEWVATISVG |
| | GGKTYYPDSVKGRFTISRDNAKNTLYLQMNSLRSEDTAMYYCAKLISLYWGQGTT |
| | LTVSS |
| 30 | EVQLQESGGGLVKPGGSLKLSCAASGFTLSSSAMSWVRQTPDRRLEWVATISVG |
| | GGKTYYPDSVKGRFTISRDNAKNTLYLQMNSLRSEDTAMYYCAKLISLYWGQGTT |
| | LTVSS |
| 31 | EVQLLESGGGLVQPGGSLRLSCAASGFTFSSSAMSWVRQAPGKGLEWVSTISVG |
| | GGKTYYPDSVKGRFTISRDNSKNTLYLQMNSLRAEDTAVYYCAKLISLYWGQGTL |
| | VTVSS |
| 32 | AVQLVESGGGLVKPGGSLKLSCAASGIIFSSSAMSWVRQTPEKRLEWVATISSGG |
| | RSTYYPDSVKGRFTVSRDSAKNTLYLQMDSLRSEDTAIYYCAKLISPYWGQGTTLT |
| | VSS |
| 33 | EVQLLESGGGLVQPGGSLRLSCAASGFTFSSSAMSWVRQAPGKGLEWVSTISSG |
| | GRSTYYPDSVKGRFTISRDNSKNTLYLQMNSLRAEDTAVYYCAKLISPYWGQGTL |
| | VTVSS |
| 34 | EVQLLESGGGLVQPGGSLRLSCAASGIIFSSSAMSWVRQAPGKGLEWVATISSGG |
| | RSTYYPDSVKGRFTVSRDSSKNTLYLQMNSLRAEDTAVYYCAKLISPYWGQGTLV |
| | TVSS |
| 35 | EVQLVESGGGLVKPGGSLKLSCAASGFTLSTHAMSWVRQTPEKRLEWVATISSG |
| | GRSTYYPDSVKGRFTISRDNVKNTLYLQLSSLRSEDTAVYFCARLISTYWGQGTTL |
| | TVSS |
| 36 | EVQLLESGGGLVQPGGSLRLSCAASGFTFSTHAMSWVRQAPGKGLEWVSTISSG |
| | GRSTYYPDSVKGRFTISRDNSKNTLYLQMNSLRAEDTAVYYCAKLISTYWGQGTL |
| | VTVSS |
| 37 | EVQLLESGGGLVQPGGSLRLSCAASGFTLSTHAMSWVRQAPGKGLEWVATISSGGR |
| | STYYPDSVKGRFTISRDNSKNTLYLQMNSLRAEDTAVYFCARLISTYWGQGTLVTVSS |
| 38 | EVQLLESGGRLVTPGGSLTLTCAASGIIFSSSAMSWVRQAPGKGLEWVATISSGGRST |
| | YYPDSVKGRFTVSRDSSKNTLYLQMNSLRAEDTAVYYCAKLISPYWGQGTLVTVSS |
| 39 | QIQLVQSGPELKKPGETVKISCKASGYTFTTYGNTWVKQAPGKGLKWMGWINTSSGV |
| | PTYADDFKGRFAFSLETSASTAYLQINNLKSEDTATYFCARPEVAYWGQGTLVTVSA |
| 40 | QVTLKESGPGILQPSQTLSLTCSFSGFSLSTSGMGVSWIRQPSGKGLEWLAHIYWDDD |
| | KRYNPSLKSRLTISKDTSSNQVFLKITSVDTADTATYYCARSGTTAPFAYWGQGTLVTV |
| | SA |
| 41 | AEELVLERCDLELETNGRDHHTADLCREKLVVRRGQPFWLTLHFEGRNYEASVDSLTF |
| | SVVTGPAPSQEAGTKARFPLRDAVEEGDWTATVVDQQDCTLSLQLTTPANAPIGLYRL |
| | SLEASTGYQGSSFVLGHFILLFNAWCPADAVYLDSEEERQEYVLTQQGFIYQGSAKFIK |
| | NIPWNFGQFEDGILDICLILLDVNPKFLKNAGRDCSRRSSPVYVGRVVSGMVNCNDDQ |
| | GVLLGRWDNNYGDGVSPMSWIGSVDILRRWKNHGCQRVKYGQCWVFAAVACTVLR |
| | CLGIPTRVVTNYNSAHDQNSNLLIEYFRNEFGEIQGDKSEMIWNFHCWVESWMTRPDL |
| | QPGYEGWQALDPTPQEKSEGTYCCGPVPVRAIKEGDLSTKYDAPFVFAEVNADVVD |
| | WIQQDDGSVHKSINRSLIVGLKISTKSVGRDEREDITHTYKYPEGSSEEREAFTRANHL |
| | NKLAEKEETGMAMRIRVGQSMNMGSDFDVFAHITNNTAEEYVCRLLLCARTVSYNGIL |
| | GPECGTKYLLNLNLEPFSEKSVPLCILYEKYRDCLTESNLIKVRALLVEPVINSYLLAERD |
| | LYLENPEIKIRILGEPKQKRKLVAEVSLQNPLPVALEGCTFTVEGAGLTEEQKTVEIPDP |
| | VEAGEEVKVRMDLLPLHMGLHKLVVNFESDKLKAVKGFRNVIIGPA |

One can easily determine whether an antibody binds to the same epitope as, or competes for binding with, another antibody by using routine methods known in the art. For example, to determine if a test antibody binds to the same epitope as a reference antibody of the invention, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention. To determine if an antibody competes for binding with a reference antibody, the above-described binding methodology is performed in two orientations. In a first orientation, the reference antibody is allowed to bind to a protein/peptide under saturating conditions followed by assessment of binding of the test antibody to the protein/peptide molecule. In a second orientation, the test antibody is allowed to bind to the protein/peptide under saturating conditions followed by assessment of binding of the reference antibody to the protein/peptide. If, in both orientations, only the first (saturating) antibody is capable of binding to the protein/peptide, then it is concluded that the test antibody and the reference antibody compete for binding to the protein/peptide. As will be appreciated by the skilled person, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50%, 75%, 90% or even 99% as measured in a competitive binding assay. Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

Any subject may be treated in accordance with the invention. The subject is preferably a human. However, the subject may be another mammalian animal, such as a non-human primate, a horse, a cow, a sheep, a pig, a dog, a cat, a rabbit, a rat, a mouse, a guinea pig or a hamster. In the context of the invention as a whole, the scleroderma disease is preferably a localized scleroderma or a systemic scleroderma. Localised scleroderma encompasses Morphea, linear scleroderma, eosinophilic fascilitis or yet toxin-induced syndromes. Systemic scleroderma encompasses limited scleroderma, diffuse scleroderma as well as overlap syndromes. The scleroderma disease can also be systemic scleroderma with interstitial lung disease. Scleroderma diseases can happen in various different tissues or organs, such as skin, lung, liver, gastrointestinal, pancreas, heart and/or kidney.

Any anti-TG2 antibody according to the invention may be incorporated into pharmaceutical compositions suitable for administration to a subject in any way, such as (but not limited to) topically, intra nasally, intradermally, intravenously, subcutaneously or intramuscularly. Typically, the pharmaceutical composition comprises the anti-TG2 antibody and one or more pharmaceutically acceptable adjuvant(s) and/or carrier(s). Therefore, herein described is also a pharmaceutical composition for use in the treatment of a scleroderma disease, such as localized scleroderma or systemic scleroderma, wherein said pharmaceutical composition comprises an anti-TG2 antibody and one or more pharmaceutically acceptable adjuvant(s) and/or carrier(s). The pharmaceutical composition can also be for use in the treatment of systemic scleroderma with interstitial lung disease. The pharmaceutical composition according to the invention can be part of a kit with instructions for use, including instructions and optionally a device for intravenous, subcutaneous or intramuscular administration to the individual in need thereof.

As used herein, “pharmaceutically acceptable carrier” includes any and all solvents, dispersion media, coatings, antibacterial and antifungal agents, isotonic and absorption delaying agents, and the like that are physiologically compatible and are suitable for administration to a subject for the methods and uses described herein. Examples of pharmaceutically acceptable carriers include one or more of water, saline, phosphate buffered saline, dextrose, glycerol, ethanol and the like, as well as combinations thereof. Depending on the route of administration or the type of formulation (such as liquid, freeze-dried or spray-dried formulation), isotonic agents can be incorporated, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The pharmaceutical compositions according to the present invention may be in a variety of forms. These include, for example, liquid solutions (e.g., injectable and infusible solutions), dispersions or suspensions, powders and liposomes. The preferred form depends on the intended mode of administration and therapeutic application. Typical preferred compositions are in the form of injectable or infusible solutions, such as compositions similar to those used for passive immunization of humans with other antibodies.

A suitable dosage of an anti-TG2 antibody according to the present invention may be determined by a skilled medical practitioner. Actual dosage levels of the active ingredients in the pharmaceutical compositions of the present invention may be varied so as to obtain an amount of the active ingredient that is effective to achieve the desired therapeutic response for a particular subject, composition, and mode of administration, without being toxic to the subject. The selected dosage level will depend upon a variety of pharmacokinetic factors including the route of administration, the time of administration, the rate of excretion of the antibody, the duration of the treatment, other drugs, compounds and/or materials used in combination with the particular antibody, the age, sex, weight, condition, general health and prior medical history of the subject being treated.

A suitable dose may be, for example, in the range of from about 0.01 pg/kg to about 1000 mg/kg body weight, typically from about 0.1 pg/kg to about 100 mg/kg body weight, of the subject to be treated. Dosage regimens may be adjusted to provide the optimum desired response (e.g. a therapeutic response). For example, a single dose may be administered, or several divided doses may be administered over time. Dosage unit form as used herein refers to physically discrete units suited as unitary dosages for the subjects to be treated; each unit contains a predetermined quantity of active compound calculated to produce the desired therapeutic effect in association with the required pharmaceutical earner. Administration may be in single or multiple doses. Multiple doses may be administered via the same or different routes and to the same or different locations.

In the context of the invention as a whole the anti-TG2 antibody may be co-administered with one or other more other therapeutic agents. Combined administration of two or more agents may be achieved in a number of different ways. Both may be administered together in a single composition, or they may be administered in separate compositions as part of a combined therapy. For example, the one may be administered before or separately, after or sequential, or concurrently or simultaneously with the other.

### DESCRIPTION OF THE FIGURES

FIG. 1: TG2 detection in serum of SSc subjects and healthy control samples, using a TG2 detection MSD. Data shown as individual data points, and the mean±SEM.

FIG. 2: TG2 detection in plasma of SSc subjects and healthy control samples, using a TG2 detection MSD. Data shown as box and whisker plot with box as 10-90 percentile, and individual values shown outside that (**=p<0.01).

FIG. 3: A) TG2 immunofluorescence staining in paraffin-embedded skin sections of SSc subjects and matched healthy individuals. Dots represent individual subjects and horizontal lines mean±standard deviation. B) TG2 immunofluorescence staining in paraffin-embedded skin sections of SSc subjects with or without SSc-associated pulmonary fibrosis. Semiquantitative evaluation ranging from 0 (no staining), over 1 (moderate staining) to 2 (intense staining). Dots represent individual subjects and horizontal lines mean±standard deviation.

FIG. 4: TG2 immunofluorescence staining of cryosections of healthy and SSc subjects. Semiquantitative evaluation ranging from 0 (no staining), over 1 (moderate staining) to 2 (intense staining). Dots represent individual subjects and horizontal lines mean±standard deviation.

FIG. 5: Comparison of TG2 expression patterns across the scleroderma disease spectrum globally and within specific dermal compartments using IA12. The expression and distribution of staining were assessed and scored between 0-3 with 0 (no staining) and 3 (maximal staining) in 0.5 increments. Data represents mean staining score ±SD from 5 biopsy samples. *=p, 0.05. ** p<0.01, ***p<0.001.

FIG. 6: TG2 antigen in cultured human dermal or lung fibroblasts derived from SSc subjects and matched healthy volunteers (NF). Data represents mean±SEM densitometry data from Western blots. *=p<0.05 (t-test).

FIG. 7: TG2 activity in cultured human dermal fibroblasts derived from SSc patient subjects and matched healthy individuals (NF) (left). TG2 activity is reduced in normal cells upon application of anti TG2 antibody BB7 (right). Dots represent individual patient subjects and horizontal lines indicate the mean.

FIG. 8: TG2 activity in cultured human dermal fibroblasts derived from SSc subjects and matched healthy individuals from a second site. Comparison between limited and diffuse cutaneous SSc also shown. Dots represent individual subjects and horizontal lines indicate the mean. **=p<0.01, ***=p<0.001 (ANOVA).

FIG. 9: TG2 activity in cultured human dermal fibroblasts derived from SSc subjects with or without pulmonary fibrosis. Dots represent individual subjects and horizontal lines indicate the mean. *=p<0.05, **=p<0.01, ***=p<0.001 (ANOVA).

FIG. 10: TG2 expression in cultured human dermal fibroblasts isolated from SSc subjects and healthy individuals. Western blotting (A) with subsequent quantification (B) of five lines from SSc subjects and five lines from respective controls. Dots represent individual subjects and horizontal lines mean±standard deviation. **=p<0.01 (t-test).

FIG. 11: Inhibitory activity of BB7 on TG2 activity in human dermal fibroblasts. Dose-titration of the anti-TG2 antibody BB7 in human dermal fibroblasts with the isotype antibody 922 as control. Dots represent individual subjects; bars represent the mean and error bars the standard deviation.

FIG. 12: Inhibitory activity of BB7 on ECM deposition in SSc-fibroblasts. CX5-based quantification of fibronectin (A) and collagen I/III (B) deposition in TGFβ1-stimulated SSc dermal fibroblasts incubated with the anti-TG2 antibody BB7 or the isotype antibody 922. Dots represent individual subjects; bars represent the mean and error bars the standard deviation. *=p<0.05 (ANOVA).

FIG. 13: Inhibitory activity of BB7 on collagen deposition in a responsive SSc-fibroblast line. CX5-based quantification of collagen (types I and III) deposition of SSc dermal fibroblasts incubated with the anti-TG2 antibody BB7 or the control isotype antibody 922. Dots represent repeat cultures of the cells from one responsive subject, bars represent the mean and error bars the standard deviation. *=p<0.05, **=p<0.01) (ANOVA).

FIG. 14: Inhibitory activity of BB7 on ECM deposition in a responsive human dermal fibroblast “line”. Dose-titration of the anti-TG2 antibody BB7 with the isotype antibody 922 as control with subsequent quantification of fibronectin (A) and type I collagen (B) deposition using CX5. Bars represent the mean and error bars the standard deviation of technical replicates. *=p<0.05 (ANOVA).

FIG. 15: Inhibitory activity of BB7 on mRNA markers of fibroblast activation and TGFβ1-Smad signalling in human dermal fibroblasts. (A) Effects of the anti-TG2 antibody BB7 and of a non-targeting control antibody (concentration 1000 nM for both) on the mRNA levels of alpha-smooth muscle actin (alphaSMA, ACTA2) and Col1a1 measured by real-time qPCR. (B) mRNA levels of the prototypical Smad target gene Ctgf and soluble collagen in the supernatant measured by real-time qPCR and SirCol assays, respectively. (C) Quantification of the mean fluorescence intensity of immunofluorescence staining for pSMAD3. (D) Expression of collagen type 1, α-smooth muscle actin(a-MA) and CTGF/CCN2 were analysed by Western blotting and normalised to expression of GAPDH. Densitometry analysis of Western blots (RHSpanels). Bars show mean±SEM. Statistical significance was tested using the T-Test, *p<0.05. In Figures A), B) and C) dots represent individual subjects, bars represent the mean and error bars the standard deviation of technical replicates.

FIG. 16: Inhibitory activity of BB7 on ECM components in SSc fibroblasts and normal fibroblasts. Images are greyscale single channel immunofluorescence images for an individual ECM component, or a composite image. Graphs represent the ratio of ECM present with and without treatment with BB7 and show that BB7 can reduce ECM in SSc fibroblasts but does not do this in normal fibroblasts.

FIG. 17: Inhibitory activity of BB7 on ECM deposition in a full-thickness skin model. The full thickness skin model is composed of human dermal fibroblasts in a three-dimensional ECM overlaid by a fully polarized dermis consisting of differentiating human keratinocytes. (A) Representative tissue sections stained with Trichrome and histological quantification of the dermal thickness. (B) Thickness of the gel on macroscopic quantification. (C) Quantification of the number of myofibroblasts. (D) Representative Western blot for Type I collagen and the house keeping protein beta-actin, (E) with normalized quantification of expression. Dots represent individual subjects; bars represent the mean and error bars the standard deviation of technical replicates. *=p<0.05, **=p<0.01 (ANOVA).

FIG. 18: TG2 deletion protects mice from bleomycin-induced histological changes in the skin. Skin thickness of WT and TG2KO mice injected intradermally with bleomycin. Dots represent individual animals and are an average of three skin thickness measurements on Masson's Trichrome or PSR stained sections. Individual p-values are shown.

FIG. 19: (A) Expression of TG2 (IA12 antibody) in primary fibroblasts from healthy controls (NF) (n=6) and scleroderma fibroblasts (n=6) was analysed by Western blotting and normalised to expression of GAPDH. (B) TG2 expression in primary fibroblasts from healthy controls (NF) (n=3) was determined following fibroblast treatment with TGFβ1 (4 mg/ml) for 24 hours.

FIG. 20: TG2 inhibition attenuates TGFβ1 induced expression of fibrotic protein markers in control and SSc dermal fibroblasts. Dermal fibroblasts isolated from healthy controls (NF; n=3) or scleroderma fibroblasts (SSc: n=1) were cultured alone or with recombinant TGFβ1 (4 ng/ml), and then treated with combinations of control IgG, antibody BB7, a pan-TGFβ1 blocking antibody or in the presence of a small molecule inhibitor of ALK5/TGF31 RI. Expression of collagen type I and α-smooth muscle actin (α-SMA) were analysed by Western blotting.

FIG. 21: Inhibitory activity of BB7 on TGFβ1 expression compared to control IgG and no treatment in primary SSc dermal fibroblasts. Data represents means luminescence ±SEM.

FIG. 22: TG2 Knock out mouse skin has thinner and less crosslinked skin under polarized light. Total percentages of green, yellow, orange and red collagen fibres under polarized light were determined in wild type and TG2 null mice following Picrosirius Red staining. The red bar at the bottom, followed by the orange bar, the yellow bar and the green bar at the top denote collagen fibers that typically represent thicker and highly crosslinked collagen fibers down to thinner collagen fibres, respectively. N=6 mice per group.

FIG. 23: TG2 deletion protects mice from bleomycin-induced collagen changes in the skin. Dermal collagen content from 4 mm dermal biopsies following bleomycin-induced skin injury in WT and TG2KO mice using SirCol assay. Dots represent individual subjects. Measurements are presented as fold-change relative to the saline treated controls (WT mice). Data is presented as fold change in collagen levels. p=0.0001**** (ANOVA).

FIG. 24: Assessment of dermal fibroblast migration using scratch-wound assay. Primary dermal fibroblasts were cultured to confluence and the monolayers scratched to induce a single injury in the monolayer. Left panel shows a 0-hour time point following wounding. Middle and right panels show scratch repair after 48 hours for WT and TG2 null fibroblast populations, respectively. The upper and lower panels are in the absence and presence of TGFβ1, respectively. The panels are in the presence of 0.5% BSA.

FIG. 25: Biomechanical force using 3-D collagen gel contraction assay. WT and TG2KO fibroblast populations derived from explant skin cultures were placed with 3-D collagen gels. The level of contraction was assessed at 48 hours in the absence or presence of TGFβ1. Contraction was determined by quantification of gel weight after contraction. *=p<0.05, ****=p<0.0001 (ANOVA).

### EXAMPLES

### Material

### Anti-TG2 Antibodies:

One anti-TG2 mAb that was used in the examples is BB7, comprised a light chain variable region as defined in SEQ ID NO:19 and a heavy chain variable region as defined in SEQ ID NO: 32.
Another anti-TG2 mAb that was used in the examples comprised a light chain variable region as defined in SEQ ID NO: 25 and a heavy chain variable region as defined in SEQ ID NO: 38. It is a rabbitised version of the original BB7, and is herein named rbBB7 or mAb1 in the following examples.

As they both have similar IC50 for human TG2 (data not shown), both were used indifferently in the following examples, depending on availability.

Zampilimab (also known as UCB7858; derived from the antibody DC1), an anti-TG2 antibody having a variable light chain according to SEQ ID NO: 24 and a variable heavy chain according to SEQ ID NO: 37 is a humanised antibody binding specifically human. In order to be able to mimic its effects on animal models, such as rabbit, mAb1 (rbBB7) has been developed. Zampilimab/DC1 and rbBB7/BB7 have been shown to behave in a similar way. They bind to the same epitope in the TG2 core (aa 313-325 of SEQ ID NO: 41), have almost identical IC50 (0.25 vs 0.3 nM) and Kd (<50 vs<60 pm) against human TG2) and inhibit ECM accumulation comparably in in vitro cell based assays. The only notable difference is the inferior IC50 of Zampilimab against rabbit TG2 (103 vs 8 nM). Therefore, the findings from the following examples using BB7/rbBB7 are fully applicable to zampilimab and any other of the anti-TG2 antibodies such as the ones herein described.

Anti-TG2 antibody IA12: the anti-TG2 mAb that was used in the following examples comprised a light chain variable region as defined in SEQ ID NO: 26 and a heavy chain variable region as defined in SEQ ID NO: 39. It is named mAb2 in the following examples. This antibody binds to an epitope that is different from the one of BB7/Zampilimab.

The antibody 922: is a control antibody binding a Clostridium Difficile toxin.

### Method

Human samples: they were obtained from the Friederich-Alexander-University Erlangen-Nuremberg and the Royal Free Hospital London.

Assessment of TG2 in Serum, plasma and Urine samples: an MSD® assay was used, using the LGC protocol. The plated were read on the MSD S1600.

Preparation of the tissue's samples: Formalin fixed, paraffin embedded skin samples were serial sectioned and stained to correlate extent of SSc/fibrosis to TG2. Staining was made to assess SSc/fibrosis as measured by collagen content and tissue transglutaminase type 2 protein expression, and enzymatic activity.

TG2 Antigen and Activity Immunohistochemistry: TG2 antigen and isopeptidase activity (ISA) was detected by immunostaining on frozen slices of lung tissue (that had been inflated with 30% sucrose at harvest and then snap-frozen), according to standard protocols.

TG2 expression: For the histology, this was scored semi-quantitatively by an experienced scientist, and for the western blot, this was determined by densitometry.

Western blot: Western blots were performed according to standard protocols. The membranes were incubated either with antibodies against TG2 (dilution of 1:100), with antibodies against β-actin (dilution of 1:5000). with antibodies against α-SMA (71 ng/mL), with antibodies against collagen type I/Col-1 (0.4 μg/mL), or with antibodies against GAPDH (0.2 μg/mL) overnight. Membranes were then incubated with the secondary antibodies for 1 hour at room temperature. Blots were revealed using enhanced chemiluminescence (ECL). For growth factor treatment cells were incubated with TGFβ (4 ng/ml) and incubated for a further 24 hours before being lysed for Western blot analysis.

TG2 MSD: TG2 in patient plasma was determined by an MSD assay according to standard protocols.

Extracellular TG activity assay: Cells were grown in 96-well plates for 7 days. Culture media was replaced with media containing 1 mM calcium, and 100 uM biotin-cadaverine. This was incubated at 37 degrees for 1 hour before being washed twice with 10 mM EDTA. Cells were then lysed with 100 μL of 0.25 mol/L ammonium hydroxide in 50 mmol/L TRIS for 5 minutes. Lysed cells were then washed with PBS and blocked with 5% BSA for 30 minutes. Blocking buffer was removed and Streptavidin-HRP added for 1 hour at RT. Wells were then washed with PBS 3 times. 100 μL TMB solution was added, and colour allowed to develop for 5-10 minutes. 50 μL stop solution was added and the absorbance read at 450 nm.

Full thickness skin equivalent (3D skin model): Fibroblasts were suspended in collagen neutralization solution (comprising DMEM, foetal calf serum, HEPES and chondroitin sulfate) and mixed with type I rat collagen. The solutions were aliquoted in transwell. After 45 minutes of incubation at 37° C. to allow for collagen polymerization, DMEM-F12 (further comprising with heat-inactivated foetal bovine serum, penicillin/streptomycin, L-glutamine and amphotericin B) was added inside each transwell on top of the collagen matrix. Keratinocytes were added the following day. Before being added, the keratinocytes were carefully detached using Accutase (Thermo Fisher Scientific), centrifuged and 0.5·10⁶ keratinocytes resuspended in E2 medium (EpiLife basal medium supplemented with Human Keratinocyte Growth Supplement, penicillin/streptomycin and CaCl2). The medium in the transwell was also replaced with E2 medium. On the next day, the medium was changed to E3 (E2 medium supplemented with 2-Phospho-L-ascorbic acid trisodium salt and keratinocyte growth factor). The medium was removed from the top of the transwells to expose the keratinocyte layer to air. From day 3 on, the E3 medium was changed every other day. In a subset of samples, TGFβ (10 ng/mL) and antibodies (BB7 or control antibodies, both at 1000 nM) were added with every medium change.

TGFβ1 assay: Primary cultures of cells derived from 4 patients with dcSSC were grown for 48 hours while being treated with either 1000 nM of BB7 or control IgG. The media was subsequently removed and the level of active TGFβ1 was measured using the mink lung cell bioassay overnight (according to standard protocol).

Scratch-wound assay: Wound closure assays were performed by injuring the monolayer of fibroblasts from either WT mice or TG2KO mice to produce cell-free lines on confluent cell monolayers with sterile plastic pipette tips. Migration of cells into the clearing space was then monitored for 48 hours and photographed as per standard protocols. Cells were treated with 0.5% BSA (negative control, no FBS) or 10% FBS (positive control), either alone or with 0.1% 2 ng/ml TGFβ1.

3-D collagen matrix contraction: Twenty-four-well tissue culture plates were precoated with sterile 2% bovine serum albumin in PBS (2 ml/well) to prevent the gels from binding to the plastic. Gel contraction assays were performed by allowing 2 ml of DMEM with 10% fetal bovine serum (FBS), 1.0 mg/ml bovine type I collagen, and 8×10⁴ fibroblasts/pericytes from WT or TG2KO mice to form a gel for 3 hours at 37° C. before releasing the gels from the dish. Contraction of the gel was quantified by loss of gel weight and decrease in gel diameter throughout a 48-hour period. The gels were then treated with DMEM containing 10% fetal calf serum (FCS) or 2 ng/ml TGFβ1 and then maintained for a period of 48 hours, allowing mechanical tension to develop. To initiate contraction, the gels were gently released from the culture dish using a sterile pipette tip and the contraction monitored throughout a 48-hour period.

Statistical analysis: Statistical significance to compare groups was calculated by one way ANOVA or unpaired student two-tailed t-test, using Microsoft Excel or GraphPad Prism V8.43. A value of p<0.05 was considered significant.

### Example 1—TG2 Expression in Subjects with Scleroderma

The objective of this study was to determine if the presence of collagen and TG2 expression were correlated with scleroderma and its severity.

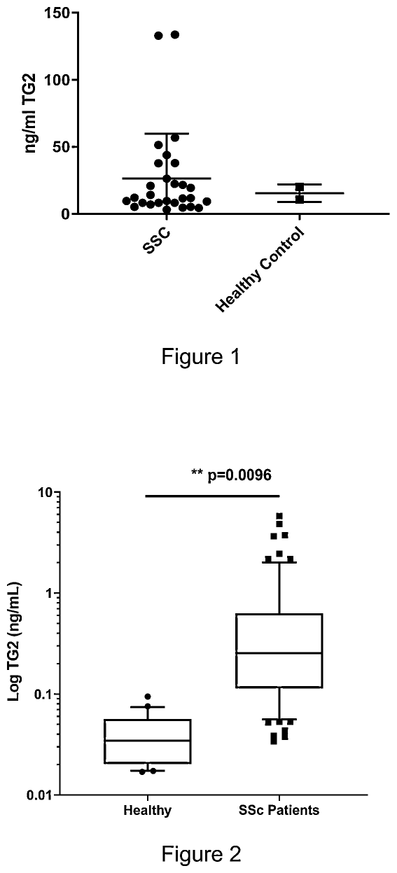

Presence of TG2 in serum samples from 200 SSc subjects and 26 healthy volunteers was assessed. TG2 was detectable in only 29 out of 200 SSc subjects and in 2 out of 26 healthy individuals (FIG. 1). TG2 positive subjects did not differ from TG2 negative subjects with regards to disease subtype, antibody profile, disease duration, disease activity, organ involvement and treatment with potentially disease modifying antirheumatic drugs.

In addition to the serum samples above analysed, presence of TG2 in plasma samples from 76 SSc subjects and 20 healthy volunteers was assessed. These samples showed a clear increase in TG2 in the plasma of SSc patients compared to controls (FIG. 2).

Then, urine samples from 30 SSc subjects and 20 healthy volunteers were analysed for detecting the presence of TG2. The protein was detectable in none of those samples.

As no clear clinical subtype was identified based on serum, plasma or urine samples, subjects were classified according to recognised clinical phenotypes, in order to include subjects:
With progressive/active disease,
With subjects with stable or regressive disease,
With subjects with established disease (i.e. >5 years)
With subjects with newly diagnosed disease (<18 months)
With major fibrotic internal organ involvement

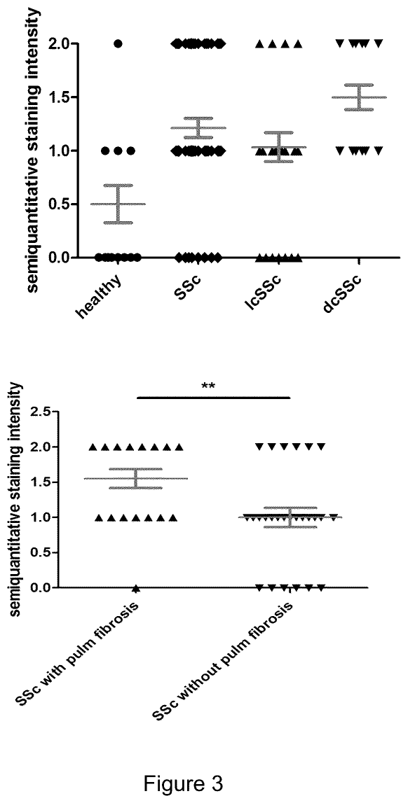

For staining, antibody IA12 was used. Sections were scored with 0 for absent staining, 1 for moderate staining or 2 for intense staining by an experienced researcher in a blinded manner. Skin sections from 56 SSc subjects and 13 controls were stained and observed for semiquantitative evaluation (FIG. 3A). Results highlighted an increased staining in SSc compared to controls. Subjects with diffuse cutaneous SSc (dcSSc) had more intense staining as compared to subjects with limited cutaneous SSc (IcSSc). Moreover, SSc subjects with pulmonary fibrosis had more intense staining than SSc subjects without pulmonary fibrosis (FIG. 3B). An elevated TG2 staining profile in inflammatory infiltrates is prominent in DcSSc non-lesional and lesional tissue (FIG. 3A). Other clinical characteristics such as disease duration, inflammatory subtype or other organ involvement were not associated with changes in TG2 expression.

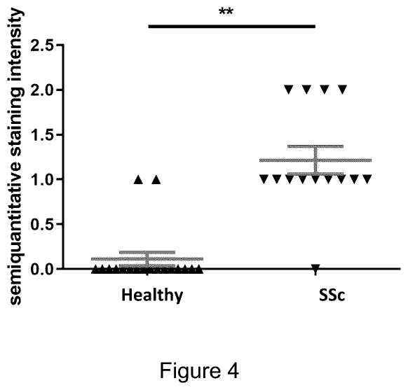

TG2 needs to be outside of the cell to both be active and be targetable with an antibody. This can be assessed using immunofluorescence for TG2 (antigen) and TG2 ISA (activity). Staining of skin cryosections from 18 healthy individuals demonstrated either no TG2 staining or a TG2 staining that was restricted to the epidermal layer of the skin with the exception of weak-to-modest staining in two subjects (FIG. 4). In contrast, staining of 14 cryosections from SSc subjects enriched for dcSSc and ILD demonstrated TG2 staining not only in the epidermis, but also in the dermis, in particular in the papillar dermis (data not shown).

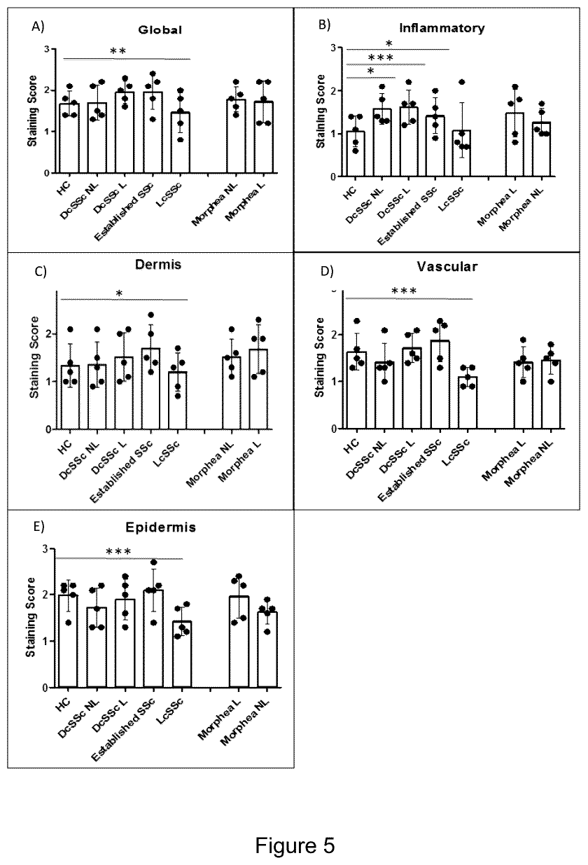

FIG. 5 aims to compare TG2 expression patterns across the scleroderma disease spectrum. Elevated profiles of TG2 expression in dermal fibroblasts were observed in the dermis from DcSSc lesional, established SSc and the two types of Morphea, however expression levels appear unaltered in LcSSc and in non-lesional DcSSc (FIG. 5C). The levels of TG2 vascular and perivascular staining remained unchanged across the scleroderma disease spectrum, except a slight increase for established SSc. A decrease in the levels of TG2 vascular staining was observed in LcSSc samples (FIG. 5D). An elevated TG2 staining profile in inflammatory infiltrates is prominent in DcSSc non-lesional and lesional tissue and in established SSc. However, staining of TG2 in inflammatory infiltrates in LcSSc appeared unaltered (FIG. 5B). TG2 expression levels in keratinocytes within the epidermal layers are evenly distributed in healthy controls (HC) and scleroderma tissue. A slight decrease in staining levels was noted in the DcSSc non-lesional samples and in LcSSc (FIG. 5E).

TG2 antigen was measured in both dermal, and lung fibroblasts from SSc patients and healthy volunteers by western blot.

### Conclusion of Example 1

These results demonstrate that TG2 is highly expressed in scleroderma lesional tissue and that within the skin TG2 is widely distributed and associated with many cells types including cell in the epidermis, and in the fibrotic dermis, fibroblasts-like cells, inflammatory cells and also closely associated with the microvasculature.

### Example 2—Intervention Studies in Primary Human Cells

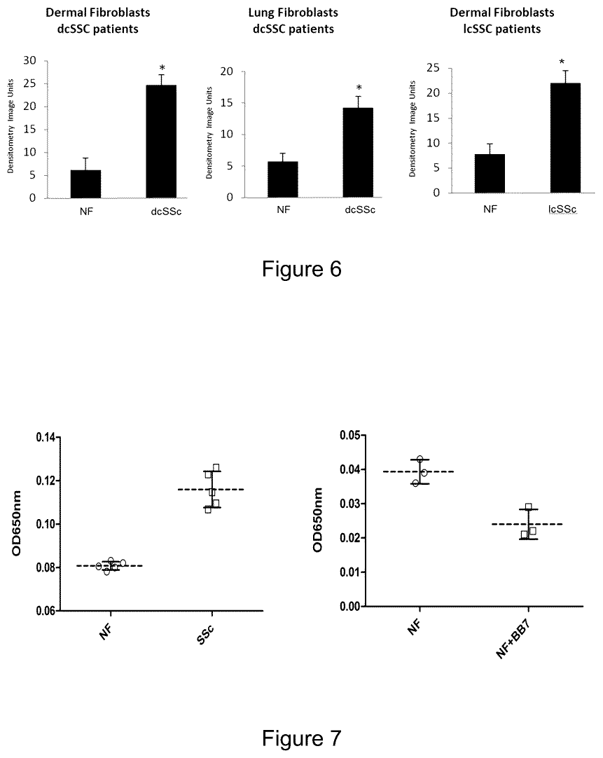

Initially, TG2 antigen was assessed in dermal and lung fibroblasts from dcSSc patients, and in dermal fibroblasts from IcSSc patients, and was shown to be elevated in patient cells compared to cells from a healthy control (FIG. 6). TG2 activity was also assessed and shown to be increased in fibroblasts from SSc patients. TG2 activity in normal fibroblasts was shown to be reduced with BB7 antibody treatment (FIG. 7).

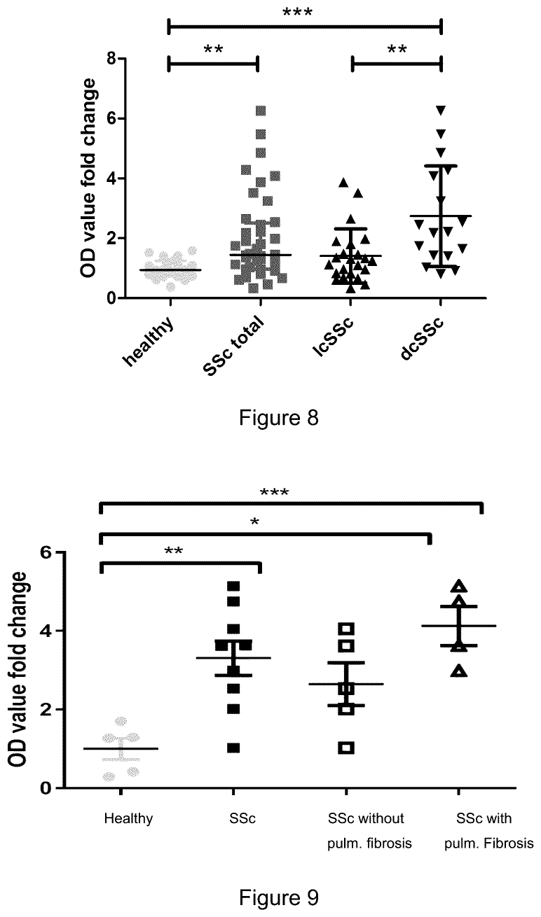

TG2 activity was then assessed in cultured human dermal fibroblasts from a second site. An increased activity of TG2 was detected in SSc fibroblasts as compared to fibroblasts from healthy individuals (FIG. 8). Further, subjects with diffuse cutaneous SSc had higher levels than subjects with limited cutaneous SSc.

Although the numbers were too small for fully accurate statistical evaluation, fibroblasts from subjects with pulmonary involvement tended to have higher levels of TG2 (FIG. 9).

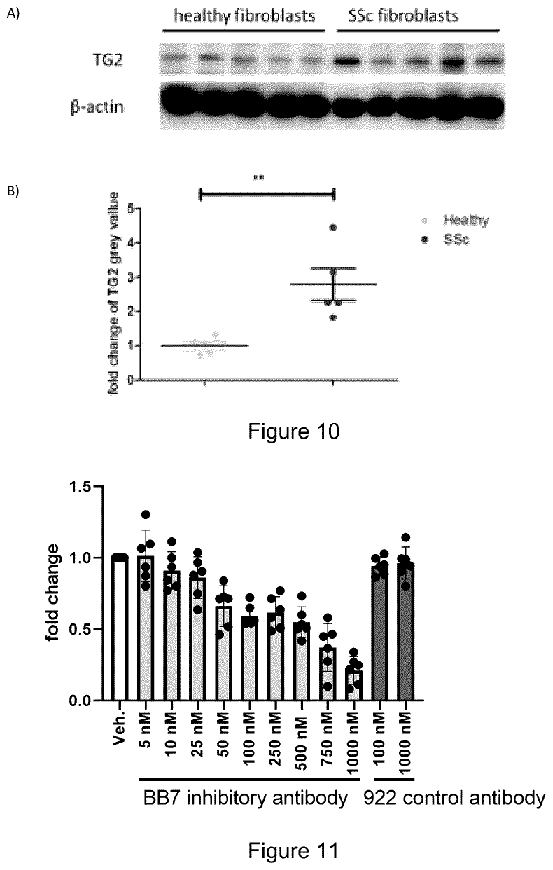

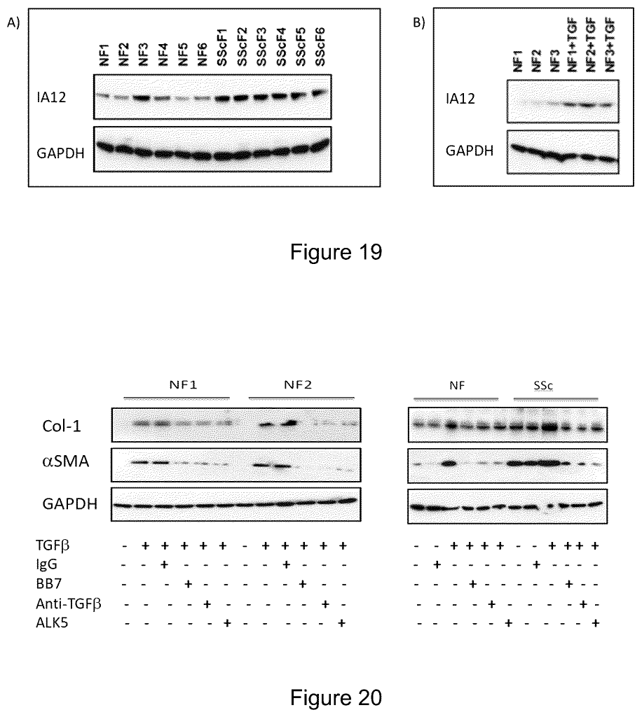

The total levels of TG2 in dermal fibroblasts derived from SSc subjects and healthy volunteers were quantified by Western blot (FIG. 10, FIG. 19A). The total levels of TG2 were increased in SSc fibroblasts compared to fibroblasts from matched healthy individuals. Pre-treatment with TGFβ1 for 24 hours resulted in an increase in TG2 in dermal fibroblasts from healthy volunteers to levels resembling that found in some of the SSc dermal cells (FIG. 19B).

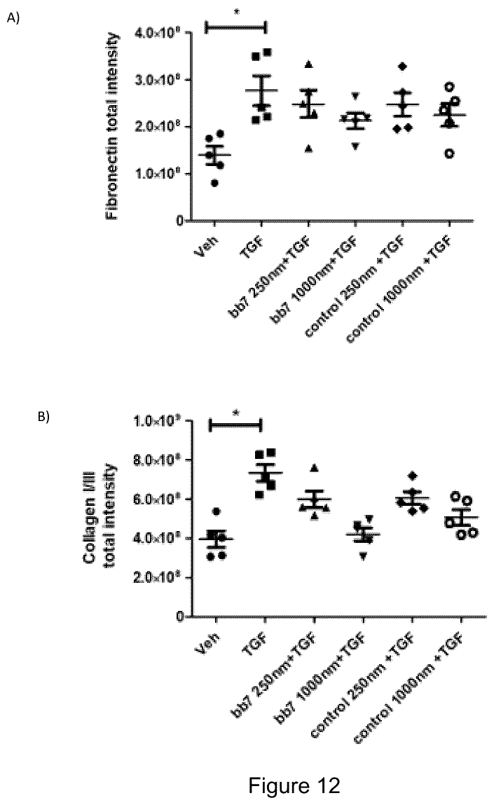

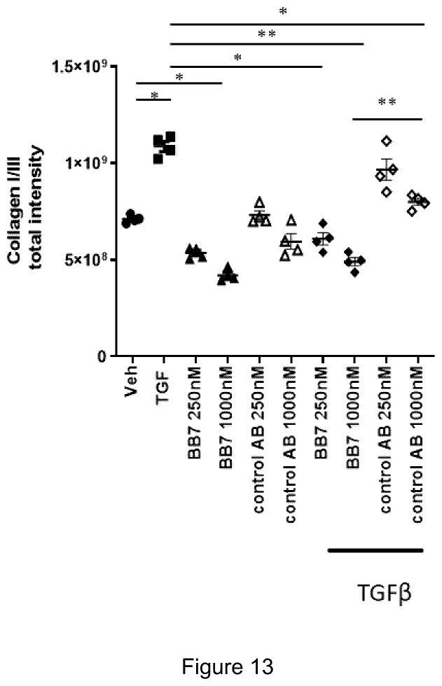

Further, the inhibitory activity of the inhibitory TG2 antibody BB7 was verified in six dermal fibroblasts lines with high levels of TG2 activity. A TG2 inhibitory antibody dose response was performed with the following antibody concentrations: 1000, 750, 500, 250, 100, 50, 25, 10 and 5 nM, in order to assess the effect said antibody on TG2 activity in the six fibroblasts lines. As demonstrated in FIG. 11, a dose-dependent reduction and observed a dose-dependent reduction of the TG2 activity with BB7, but not with the control antibody 922. Comparison of mean matrix deposition (collagen I/Ill and fibronectin) in fibroblast lines incubated with TGFβ and with the anti-TG2 antibody BB7 in concentrations of 250 and 1000 nM did not demonstrate constant and statistically differences to cells incubated with control antibodies in the same concentrations across 5 different lines (FIG. 12). It is noteworthy that some fibroblast lines incubated with control IgGs demonstrated mild inhibitory effects on fibronectin and collagen I/Ill deposition in particular at doses of 1000 nM.

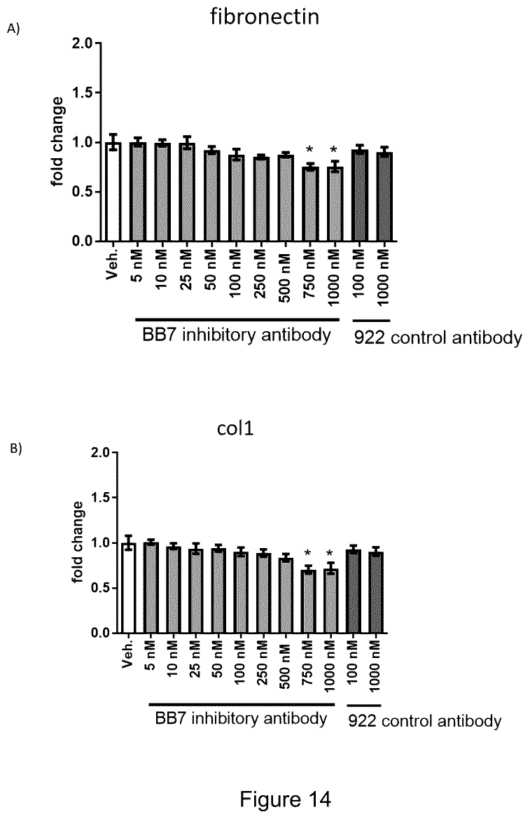

Analyses of individual fibroblast “lines” showed that TG2 inhibition strongly reduced fibronectin and collagen I/Ill deposition (FIG. 14). No differences were observed between anti-TG2 antibodies and control antibodies in the same concentrations for Collagens 2, 4, 5 and 6 (data not shown).

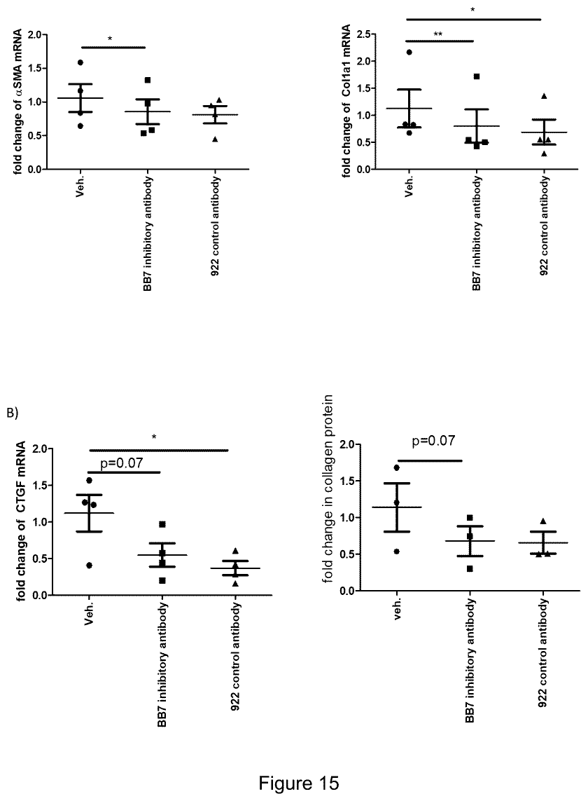

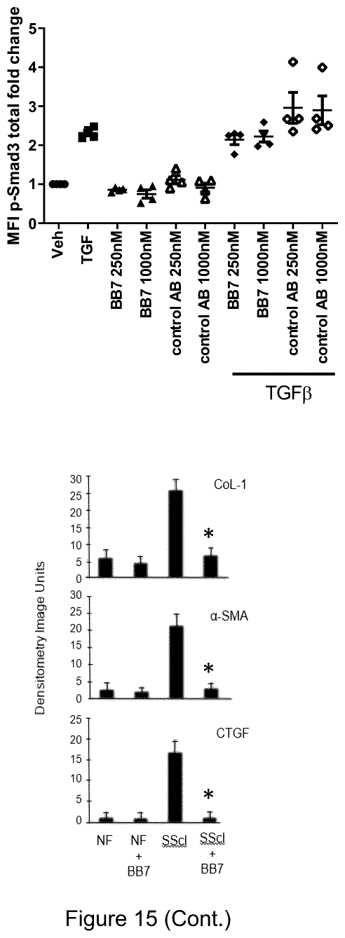

The most anti-TG2 responsive cell line was used for dose titration studies and for measuring changes in phosphorylated SMAD 2/3 and cell production of SMA, Collagen 1 and CTGF (FIGS. 14-15). No differences in the levels of pSMAD2/3 were detected in fibroblast-lines incubated with anti-TG2 antibody as compared to control antibody (FIG. 15C). TG2 inhibition reduced neither the basal levels of pSMAD2/3 nor did it reduce TGFβ1-induced accumulation of pSMAD2/3 (FIG. 15C). Further no decreases in CTGF mRNA were detected in fibroblasts incubated with anti-TG2 antibodies as compared to control antibody (FIG. 15B). However, a mild downregulation of CTGF mRNA was observed with the control antibody. Similar to the results for CTGF mRNA, the levels of COL1A1 mRNA and ACTA2 mRNA also did not differ between fibroblasts incubated with anti-TG2 antibodies as compared to control antibody (FIG. 15A). As for CTGF, control antibody induced a mild reduction in COL1A1 and ACTA2 mRNA.

Although no strong effect was observed at the mRNA level, the results highlighted in FIG. 15D show that treatments with BB7 resulted in a dramatic and significant reduction in the expression of Col-1, α-SMA and CTGF protein by scleroderma fibroblasts. The level of protein expression of the three markers in scleroderma-derived fibroblasts was found to be decline to between 70-90% of that present in the absence of BB7.

Treatment with BB7 also resulted in a reduction in the expression of Col-1 and α-SMA when dermal fibroblasts have been cultured with TGFβ1 (FIG. 20). The reduction was in a similar manner to the pan TGFβ1 blocking antibody and small molecule inhibitor of ALK5. The reduction was apparent in fibroblasts obtained from healthy control (NF) and in scleroderma fibroblasts. Compared to the fibroblasts from the healthy controls, the scleroderma fibroblasts displayed a notable basal level of Col-1 and α-SMA without any addition of TGFβ1 (FIG. 20B). Treatment with the IgG control had no effect on ether the control or scleroderma fibroblasts.

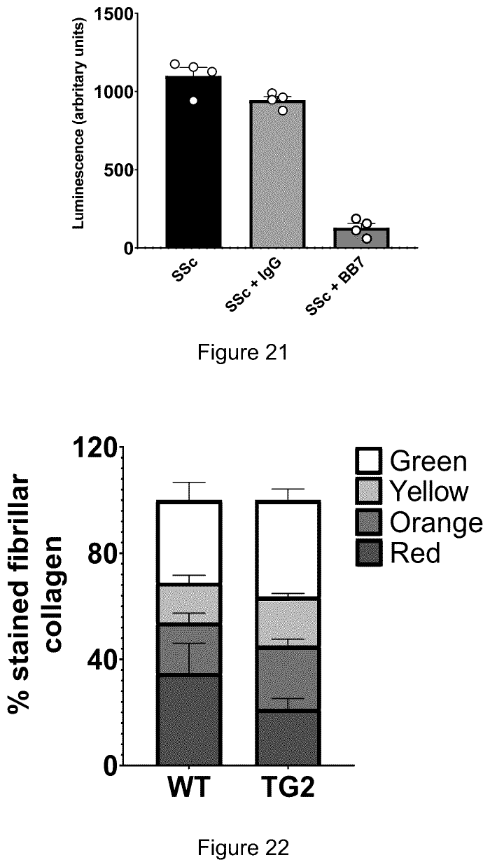

These surprising findings were indicative of a change in TGFβ1 driven SMAD signalling by blocking TG2 activity. To confirm this the levels of active TGFβ1 were measured in the media of cells exposed to BB7 using the mink lung bioassay and shown to be reduced by >80% (FIG. 21).

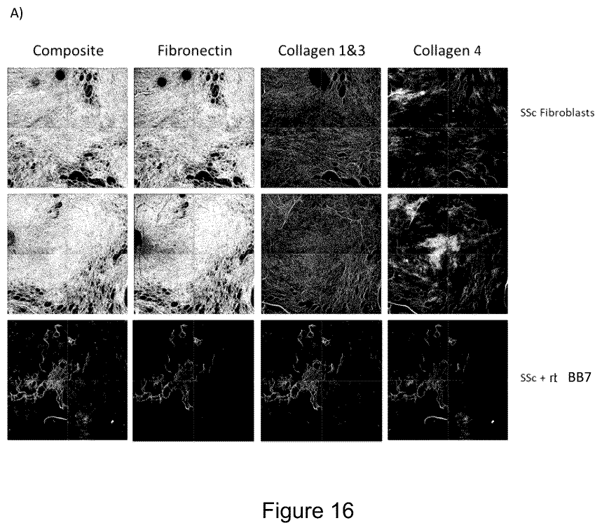

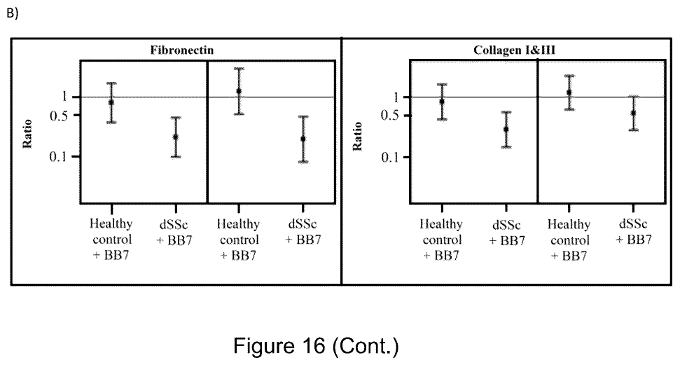

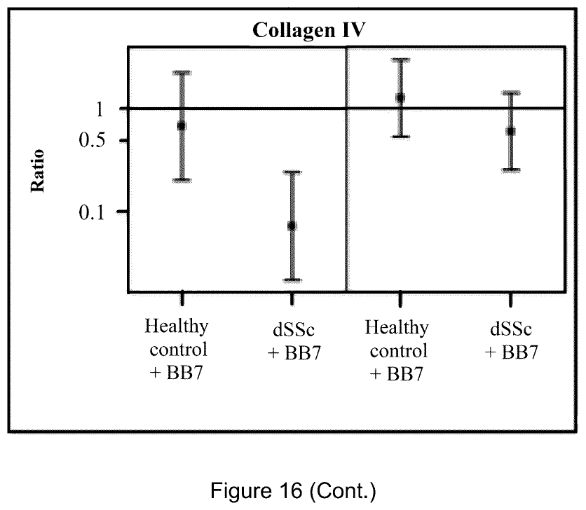

Individual ECM components were stained for using immunofluorescence techniques and quantified using automated image analysis. Fibronectin, collagens 1&2 (analysed together) and collagen IV were all reduced with application of BB7 in SSc fibroblasts, but not in healthy fibroblasts (FIG. 16).

Experiments were then reproduced on a skin composite model. Primary cultures from an identified clinical phenotype where TG2 appears to play a significant role were used for such a modelling. In this so called full-thickness skin model, fibroblasts are typically grown in a three-dimensional collagen matrix. The dermis-like part is then overlaid by epidermal keratinocytes, which are induced to undergo differentiation and polarization, which generates within a week a fully polarized epidermis that is separated from the dermis by a functional basal membrane. SSc fibroblasts as well as fibroblasts from healthy individuals can be used. A major advantage of this model besides the opportunity to study crosstalk between the two major cell populations in the skin is that fibroblasts are not per se pre-activated by the stiff plastic surfaces of conventional culture dishes.

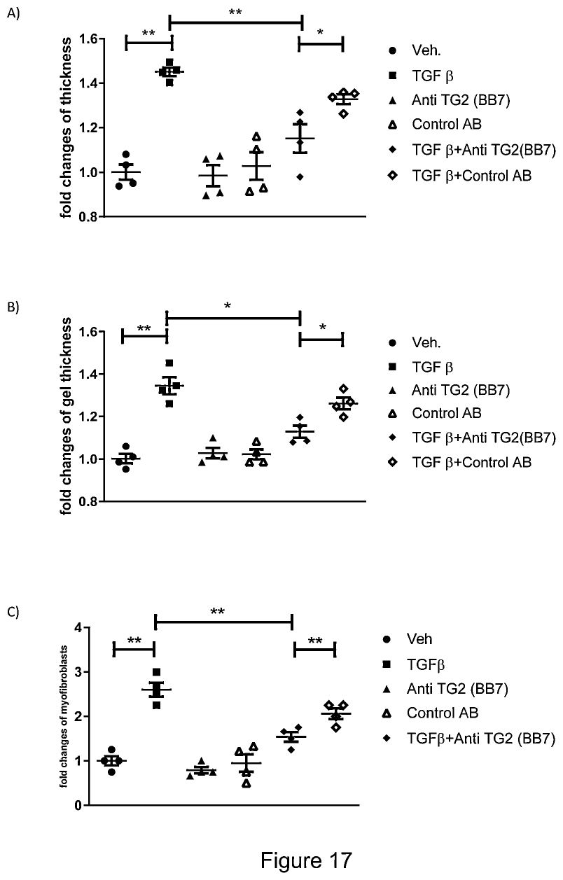

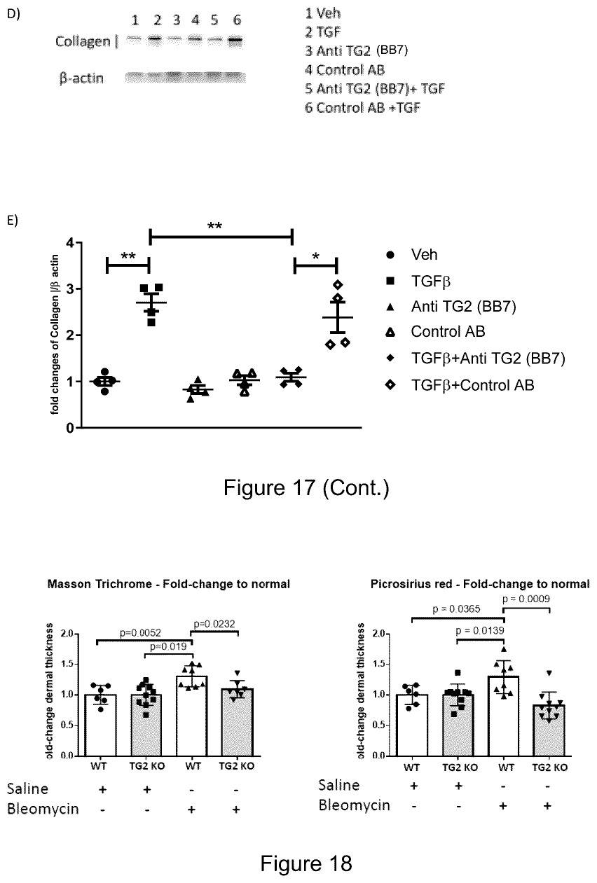

Given the mode of action of TG2 as a cross linking enzyme, it was hypothesized that this mode of action may be more relevant in a 3-dimensional culture setting that better resembles the physiologic environment of fibroblasts in the skin than standard 2D culture of fibroblasts on stiff plastic surfaces. Four different fibroblast “lines” were tested. Fibroblast “lines” were pretested in 2D and only lines with at least some mild response to TG2 inhibition were selected for further validation in the full thickness skin model. These four “lines” included the two fibroblast “lines” reported above with mild-to-moderate responses, plus two additional “lines” that were identified by screening of another six SSc “lines” (4 out of 11 screened lines). A significant antifibrotic effects was observed in presence of anti-TG2 antibody (at 1000 nM), with statistically significant decreases in TGFβ1-induced gel thickening, dermal thickening, myofibroblast counts and collagen I deposition was compared to untreated skin equivalents and compared to skin equivalents incubated with control antibodies at 1000 nM (FIG. 17). Mild effects of control antibodies were also observed in this experimental setting.

### Conclusion of Example 2

Targeting TG2 inhibition was found promising with regard to ECM deposition in a subset of SSc fibroblast “lines” in conventional 2D culture systems and in full-thickness 3D skin models. The effects were more pronounced in the full-thickness skin model, indicating that standard cell culture approaches may not be optimal to evaluate the therapeutic potential of TG2 inhibition. These antifibrotic effects seem to be independent of the TGFβ1/SMAD signalling.

### Example 3—TG2 Knockout Protects in a Model of Skin Fibrosis

To investigate the effect of TG2 deletion on the development of dermal fibrosis, the extent of bleomycin-induced skin remodelling was examined in global TG2 KO mice. For that purpose, wildtype (WT) or transglutaminase 2 knockout (TG2KO) mice were injected with either saline (Control) or 50 μL of 2 U/mL bleomycin (Bleo) intradermally into a 1 cm×1 cm patch of skin on their back, every other day for 28 days. After 28 days the animals were terminated and the skin taken for; histology, collagen assay, TG2 crosslink quantification.

Sections of skin were stained with Masson's Trichrome and Picrosirius Red staining to visualise ECM and collagens respectively. Dermal thickness measurements were then taken, with an average of three measurements across the section. The sections stained with Picrosirius Red were then placed under polarized light to visualise collagen thickness.

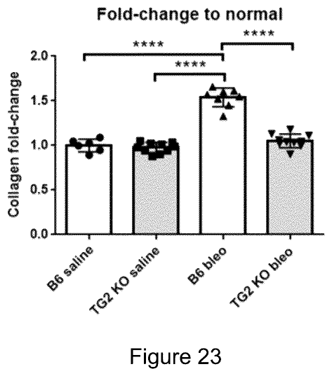

WT animals—those with a normal level of TG2—had a significant increased skin thickness (as represented by the collagen staining) in response to the bleomycin injury (FIG. 18). In contrast, the fibrotic response of the TG2 null mouse was clearly attenuated with no significant differences or changes in dermal collagen content observed between the TG2 null mice treated with saline or bleomycin. The skin thickness findings were confirmed by measured dermal collagen content in response to the bleomycin injury (FIG. 23). WT animals challenged with bleomycin had a significantly higher increase in dermal collagen compared to WT and TG2KO mice injected with saline and WT mice challenged with bleomycin.

When viewed under polarized light, thick collagen fibers appear red, and then progressively thinner through orange, yellow to the thinnest collagen fibers appearing green. The percentage distribution of collagen fiber thickness was calculated (FIG. 22). WT skin appeared predominantly red and orange, meaning a higher percentage of thick collagen fibers. The TG2KO mouse skin was predominantly green and yellow, meaning a higher percentage of thin collagen fibers. This observation was confirmed using scanning electron microscopy where the thicker “bright” collagen fibers that are most common in WT skin are all but absent in the TG2KO mice (data not shown). TG2KO mice when challenged with bleomycin does not adopt the dermal changes as seen in WT mice challenged with bleomycin.

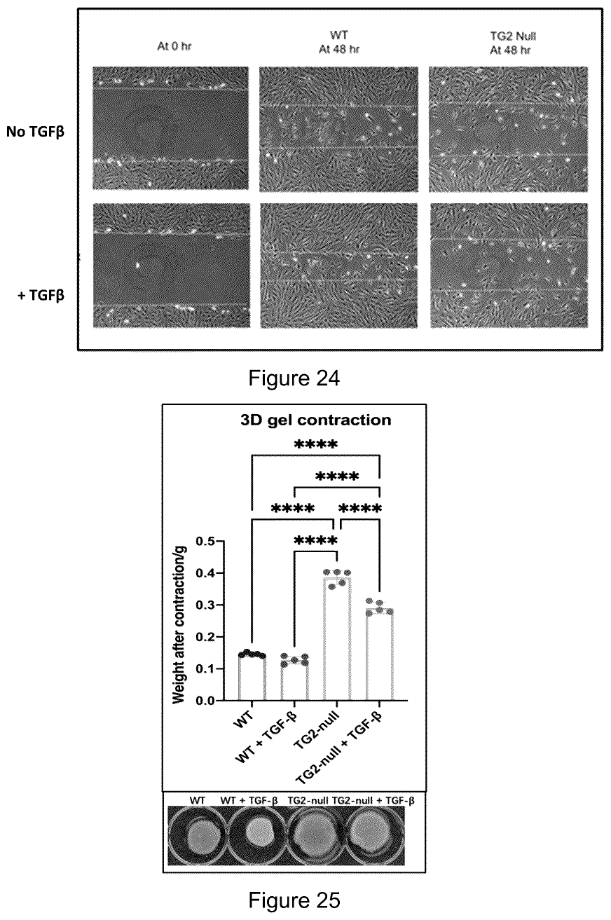

The effect of TG2 deletion was also functionally examined using a scratch test to study the migration of fibroblasts and the response to TGFβ1 (FIG. 24). Following the injury to the fibroblast monolayer, WT fibroblasts were observed to move into the wound gap and start to repair the scratch-wound from 6 hours with a substantial number of cells present within the wound site at 48 hours. Only a few TG2KO fibroblasts were observed to enter the wound site during the same 48 hour period. The addition of TGFβ1 did not affect the migration of TG2KO fibroblasts. TG2KO cells exhibit a reduced migratory capacity than WT cells when cultured in vitro.

The ability of dermal WT and TG2KO fibroblast populations to contract was explored using the 3-D type I collagen gel contraction assay (FIG. 25). TGFβ1 stimulates fibroblasts to contract relaxed collagen gels, a process that depends on the traction of migrating cells and their differentiation in contractile fibroblasts (myofibroblasts). The WT group contracted significantly more than the TG2KO group when incubated with the carrier alone (P<0.0001). The gel contraction of the TG2KO cells incubated in the presence of TGFβ1 was significantly more than that of the TG2KO cells incubated with the carrier alone (P<0.0001). This indicates that the knockout of the TG2 gene significantly affected the gel contraction, confirming that cell migration and differentiation was impaired, and by adding exogenous TGFβ1 the fibroblast migratory function could be restored and the pro-contractile behaviour rescued.

The absence of TG2 expression by fibroblasts resulted in several functional deficits crucial to scarring and fibrosis, as demonstrated by the impact on cell migration and the ability of TG2 null cells to remodel type I collagen 3-D matrices. The significantly reduced migration of these fibroblasts following scratch wounding and inability to effectively contract collagen gels suggest alterations in cell adhesion, attachment and motility, and an impairment in the transition of fibroblasts to their activated contractile myofibroblasts counterparts. These surprising findings increase the understanding of the role of TG2 in fibrogenesis and scleroderma.

### OVERALL CONCLUSION

These studies provide good evidence for a link between TG2 expression and dermal fibrosis and some insight into the potential molecular mechanism(s) involved. Together they suggest value in the TG2 inhibition, such as with anti-TG2 antibodies, as shown herein, as a promising avenue for effective therapy for connective tissue fibrosis. The data adds evidence to support the association of TG2 with fibrotic pathologies and provides further clues to the potential role of TG2 in promoting tissue fibrosis in scleroderma.

### REFERENCES

Siegel & Khosla (2007), Pharmacol. Ther., 115(2): 232-245
Wang et al. (2020) 3 Biotech., 10:287
WO2006100679
WO2012146901
WO2013175229
Careta & Romiti (2015), An. Bras. Dermatol., 90(1):62-73.
Denton & Khanna (2017), Lancet, 390: 1685-99
Barsotti et al. (2019), Clin. Exp. Rheumatol., 37 (Suppl. 119):S3-S14
WO2013175229
WO2015197772

1. An anti-Transglutaminase type 2 (TG2) antibody for use in the treatment of a subject having a scleroderma disease or in the prevention of the development of a scleroderma disease.

2. The anti-TG2 antibody for use according to claim 1, wherein the scleroderma disease is localized scleroderma, systemic scleroderma or systemic scleroderma with interstitial lung disease.

3. The anti-TG2 antibody for use according to claim 1, wherein the scleroderma disease is characterised by an increase of a marker in a subject's sample, wherein the marker is for instance any one of TG2 expression or TG2 activity.

4. The anti-TG2 antibody for use according to claim 1, wherein said antibody binds to an epitope within the core region of human transglutaminase type 2 (TG2) and inhibits human TG2 activity, wherein said core region consists of amino acids 143 to 473 of human TG2, and wherein the human TG2 activity that is inhibited is the TG2 cross-linking of lysine and glutamine with Nε(γ-glutamyl)lysine isopeptide bonds.

5. The anti-TG2 antibody for use according to claim 1, wherein the antibody or antigen-binding fragment thereof:
a. comprises or consists of an intact antibody, or
b. comprises or consists of an antigen-binding fragment selected from the group consisting of: an Fv fragment (for example a single chain Fv fragment or a disulphide-bonded Fv fragment); a Fab fragment; and a Fab-like fragment (for example an Fab′ fragment or an F(ab)2 fragment).

6. The anti-TG2 antibody for use according to claim 1, wherein said antibody comprises the following sequences:
```
(i)
(LCDR1; SEQ ID NO: 1)
KASQDINSYLT;
(LCDR2; SEQ ID NO: 2)
LVNRLVD;
(LCDR3; SEQ ID NO: 3)
LQYDDFPYT;
(HCDR1; SEQ ID NO: 4)
THAMS;
(HCDR2; SEQ ID NO: 5)
TISSGGRSTYYPDSVKG;
and
(HCDR3; SEQ ID NO: 6)
LISTY;
or
(ii)
(LCDR1; SEQ ID NO: 1)
KASQDINSYLT;
(LCDR2; SEQ ID NO: 7)
LTNRLMD;
(LCDR3; SEQ ID NO: 8)
LQYVDFPYT;
    (HCDR1; SEQ ID NO: 9)
SSAMS;
  (HCDR2; SEQ ID NO: 5)
TISSGGRSTYYPDSVKG;
and
(HCDR3; SEQ ID NO: 10)
LISPY;
or
(iii)
(LCDR1; SEQ ID NO: 1)
KASQDINSYLT;
(LCDR2; SEQ ID NO: 11)
RTNRLFD;
(LCDR3; SEQ ID NO: 3)
LQYDDFPYT;
(HCDR2; SEQ ID NO: 9)
SSAMS (HCDR1;
TISVGGGKTYYPDSVKG;
and
      (HCDR3; SEQ ID NO: 12)
LISLY.
```

7. The anti-TG2 antibody for use according to claim 1, wherein the antibody comprises:
a) a light chain variable domain having the sequence as defined in any one of SEQ ID NO: 13 to SEQ ID NO: 27 and a heavy chain variable domain having the sequence as defined in any one of SEQ ID NO: 28 to SEQ ID NO: 40,
b) a light chain variable domain having at least 80% identity or similarity, preferably at least 90% identity or similarity, or preferably at least 95% identity or similarity to the sequence as defined in any one of SEQ ID NO: 13 to SEQ ID NO: 27 and a heavy chain variable domain having at least 80% identity or similarity, preferably at least 90% identity or similarity, or preferably at least 95% identity or similarity to the sequence as defined in any one of SEQ ID NO: 28 to SEQ ID NO: 40.

8. The anti-TG2 antibody for use according to claim 1, wherein said antibody competes for binding to TG2 with an antibody comprising the following sequences:
(i)
(LCDR1; SEQ ID NO: 1)
KASQDINSYLT;
(LCDR2, SEQ ID NO: 2)
LVNRLYD;
(LCDR3; SEQ ID NO: 3)
LQYDDFPYT;
(HCDR1, SEQ ID NO: 4)
THAMS;
(HCDR2; SEQ ID NO: 5)
TISSGGRSTYYPDSVKG;
and
(HCDR3; SEQ ID NO: 6)
LISTY;
or
(ii)
(LCDR1; SEQ ID NO: 1)
KASQDINSYLT;
(LCDR2; SEQ ID NO: 7)
LTNRLMD;
(LCDR3; SEQ ID NO: 8)
LQYVDFPYT;
(HCDR1; SEQ ID NO: 9)
SSAMS;
(HCDR2; SEQ ID NO: 5)
TISSGGRSTYYPDSVKG;
and
(HCDR3; SEQ ID NO: 10)
LISPY;
or
(iii)
(LCDR1: SEQ ID NO: 1)
KASQDINSYLT;
(LCDR2; SEQ ID NO: 11)
RTNRLFD;
(LCDR3; SEQ ID NO: 3)
LQYDDFPYT;
(HCDR2; SEQ ID NO: 9)
SSAMS (HCDR1);
TISVGGGKTYYPDSVKG;
and
(HCDR3; SEQ ID NO: 12)
LISLY.

9. A method for treating a subject having a scleroderma disease or for preventing the development a scleroderma disease in a subject comprising administering a therapeutically effective amount of an anti-TG2 antibody to said subject.

10. Use of an anti-Transglutaminase type 2 (TG2) antibody for the manufacturing of a medicament in the treatment of a subject having a scleroderma disease or for the prevention of the development of a scleroderma disease in a subject.
